SEQUENCE LISTING (23 sequences)

SEQ ID NO: 1 - protein, 468 aa, Artificial Sequence
  OTA hydrolyzing polypeptide 1
MSDHLPADVRDLIRQTMSRLENLVPPSAAFDDETQDAVFEAIDSIDGDMLALNKAIHDNPELGFHEVKAHHHLVQALIKLGFAVTNPTSLPTAFVATWSHGSGGRTFGFNAEYDALPGVGHACGHNLIAIVAVASAAGLKAALKARNIPGTIKVIGSPAEEGGGGKVILLNEGAYDGIDACVMAHPEGGVPGIDDDGSAYTSGPATLARAGFSVELFGKAAHAGAAPWLGVNALDAAVQGYTAVSMLRQQIEPTMRVHGVFDYGKEKWVQNIIPAYAKVEYSTRALDVKSTLELRKKVISCFESAATATGCTHKVSAPETEVYAELRNNVTLADAYADFMTTAFGQKIGRVGITTASTDFGNVTYKMPAIHPGFHLISKGVNHTPEFADAAGTLDAHERAIKVAKGLAMLGAKFLQDDKFAKEVKRKFDEFKKEVGELKLTDNENEVQEVNGVMKQEHIAEQVDEIWL

SEQ ID NO: 2 - protein, 436 aa, Artificial Sequence
  OTA hydrolyzing polypeptide 2
MARDEIPADVKALIAQTVTALHGLAPPSAEFDDETQDAVFEAIDAIDGAMWDLNKKIHDHPELGFKEHKARANLVAALCKLGFTVTNPSSLPTAFVATYTQGHGGRTFGVDAEYDALPEVGHACGHNLIAVAAVAAAAGLKAALKARNIAGTIKLIGSPAEEGGGGKVILLNEGVYDCLDACVMAHPEGGMIGNDYDGSAYVGGPASLARAGFEIEFHGRGAHAGAAPWLGINALDAAVQGYTAVSMLRQQLEPTMRVHGIIQGSEKWAQNIIPSYAKVSYSTRALDVKKTLELRDKTVDCFASAAKATGCTYELTAPETEVYAELRNNEPLADAYADLMTHAFGQKIERRGMTTASTDFGNVTYHVPAIHPGFTIESDTVNHTAGFTEAAGKKGAHERTMKVAKGLAMVGAKFLADDKFAKDVRKAFEKFKKGAQ

SEQ ID NO: 3 - protein, 436 aa, Artificial Sequence
  OTA hydrolyzing polypeptide 3
MARDEIPADVKALIAQTVTALHGLAPPSAEFDDETQDAVFEAIDAIDDAMWDLNKKIHDHPELGFEEHKARANLVAALCKLGFTVTNPSSLPTAFVATYTHGHGGRTFGVDAEYDALRDVGHACGHNLIAVAAVAAAAGLKAALKARNISGTVKLIGSPAEEGGGGKVILLNEGVYDCLDACVMAHPEGGMIGDDYDGSAYVGGPASLARAGFEIEFHGRGAHAGAAPWLGINALDAAVQGYTAVSMLRQQLEPTMRVHGIIQGSEKWAQNIIPSYAKVSYSTRALDVKKTLELRDKTVDCFASAAKATGCTYDVTAPETEVYAELRNNQPLADAYADLMTKAFGQKIERRGMTTASTDFGNVTYHLPAIHPGFTIESETVNHTKGFTEAAGKKDAHERTMKVAKGLAMVGAKFLADDKFAKDVRKAFEKFKREVE

SEQ ID NO: 4 - protein, 439 aa, Artificial Sequence
  OTA hydrolyzing polypeptide 4
MARDPLPADVRLLIRNNMASAHPQVPTIDGLDSKTQNDVFTAIDAINNDMRDLSLAIHENPELGFKEVKAHANLVKALSKLGFTITNPTSLETAFVATWSNGSGGRTFGFNAEYDALPGIGHACGHNLIAIVAVASAVGCKAAMEKQGISGTIKVIGSPAEEGGGGKIILFNEGVYDGLDACAMAHPGGGMPADGYDGQAPAEGKPASLARAGFIVDFVGKPAHAGAAPWMGVNAVDAAVLGYNGVSMLRQQLEPTVRVHGVIRGPNDWVNNIIPASSHVEYDVRAPDVKQAMAVRNKAIACFESAAHATGCSHTTQSKDKDIYAELRNSIRMAGVYADIMEEVFDQHIQRDGMTTASTDFGNITISACPAVHPMYLIETDGVNHTPEFTAGAATMDSHERSIKVAKGLALLGFKVLTDDVFAKKIKDEFEEQKKRLGA

SEQ ID NO: 5 - protein, 10 aa, Artificial Sequence
  conservative motif
GHACGHNLIA

SEQ ID NO: 6 - protein, 12 aa, Artificial Sequence
  conservative motif
IGSPAEEGGGGK

SEQ ID NO: 7 - protein, 4 aa, Artificial Sequence
  conservative motif
MAHP

SEQ ID NO: 8 - protein, 8 aa, Artificial Sequence
  conservative motif
AHAGAAPW

SEQ ID NO: 9 - protein, 7 aa, Artificial Sequence
  conservative motif
KVAKGLA

SEQ ID NO: 10 - protein, 15 aa, Artificial Sequence
  conservative motif
IGSPAEEGGGGKVIL

SEQ ID NO: 11 - protein, 15 aa, Artificial Sequence
  conservative motif
IGSPAEEGGGGKVIL

SEQ ID NO: 12 - protein, 10 aa, Artificial Sequence
  conservative motif
PELGFHEVKA

SEQ ID NO: 13 - protein, 13 aa, Artificial Sequence
  conservative motif
TNPTSLPTAFVAT

SEQ ID NO: 14 - protein, 14 aa, Artificial Sequence
  conservative motif
GGRTFGFNAEYDAL

SEQ ID NO: 15 - protein, 17 aa, Artificial Sequence
  conservative motif
GHACGHNLIAIVAVASA

SEQ ID NO: 16 - protein, 26 aa, Artificial Sequence
  conservative motif
IPGTIKVIGSPAEEGGGGKVILLNEG

SEQ ID NO: 17 - protein, 11 aa, Artificial Sequence
  conservative motif
DACVMAHPEGG

SEQ ID NO: 18 - protein, 21 aa, Artificial Sequence
  conservative motif
AHAGAAPWLGVNALDAAVQGY

SEQ ID NO: 19 - protein, 70 aa, Artificial Sequence
  conservative motif
  misc_feature (11)..(12): Xaa can be any naturally occurring amino acid
  misc_feature (16)..(16): Xaa can be any naturally occurring amino acid
  misc_feature (18)..(18): Xaa can be any naturally occurring amino acid
  misc_feature (20)..(20): Xaa can be any naturally occurring amino acid
  misc_feature (24)..(28): Xaa can be any naturally occurring amino acid
  misc_feature (30)..(30): Xaa can be any naturally occurring amino acid
  misc_feature (33)..(33): Xaa can be any naturally occurring amino acid
  misc_feature (35)..(35): Xaa can be any naturally occurring amino acid
  misc_feature (48)..(48): Xaa can be any naturally occurring amino acid
  misc_feature (51)..(51): Xaa can be any naturally occurring amino acid
  misc_feature (55)..(55): Xaa can be any naturally occurring amino acid
  misc_feature (58)..(59): Xaa can be any naturally occurring amino acid
  misc_feature (63)..(63): Xaa can be any naturally occurring amino acid
  misc_feature (68)..(68): Xaa can be any naturally occurring amino acid
GHACGHNLIAXXAVAXAXGXKAAXXXXXIXGTXKXIGSPAEEGGGGKXILXNEGXYDXXDACXMAHPXGG

SEQ ID NO: 20 - protein, 5 aa, Artificial Sequence
  M20 peptidase aminoacylase 1-like protein 2-like amidohydrolase subfamily conservative motif
  MISC_FEATURE (5)..(5): Xaa can be Lys or His
CHEHX

SEQ ID NO: 21 - protein, 38 aa, Artificial Sequence
  M20 peptidase aminoacylase 1-like protein 2-like amidohydrolase subfamily conservative motif
  misc_feature (2)..(2): Xaa can be any naturally occurring amino acid
  misc_feature (4)..(37): Xaa can be any naturally occurring amino acid
CXHXXXXXXXXXXXXXXXXXXXXXXXXXXXXXXXXXXE

SEQ ID NO: 22 - protein, 6 aa, Artificial Sequence
  M20 peptidase aminoacylase 1-like protein 2-like amidohydrolase subfamily conservative motif
  MISC_FEATURE (6)..(6): Xaa can be Lys or His
CHEEHX

SEQ ID NO: 23 - protein, 4 aa, Artificial Sequence
  conservative motif
VNHT

(19) United States
(12) Patent Application Publication
(10) Pub. No.: US 2024/0067950 A1
(43) Pub. Date: Feb. 29, 2024

(54) MEANS AND METHODS FOR DETOXIFYING OCHRATOXIN A

(71) Applicant: DSM Austria GmbH, Getzersdorf (AT)

(72) Inventors: Shreenath PRASAD, Tulln an der Donau (AT); Christoph GONAUS, Wien (AT); Wulf-Dieter MOLL, Stockerau (AT); Gerd SCHATZMAYR, Tulln an der Donau (AT)

(21) Appl. No.: 18/268,189

(22) PCT Filed: Dec. 17, 2021
(86) PCT No.: PCT/EP2021/086480

§ 371 Date: Jun. 16, 2023

(30) Foreign Application Priority Data
Dec. 18, 2020 (EP) 20215495.1

(51) Int. Cl. C12N 9/80; A61P 39/02

(52) U.S. Cl. CPC C12N 9/80; A61P 39/02; C12Y 305/01014; A61K 38/00

(57) ABSTRACT

The present invention relates to novel polypeptides capable of detoxifying Ochratoxin A (OTA) and methods (e.g., for detoxifying mycotoxins) based thereon. The present invention further relates to compositions, kits, transgenic plants, transgenic seeds, transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof comprising one or more of the polypeptides capable of detoxifying Ochratoxin A (OTA).

### CROSS-REFERENCE TO RELATED APPLICATIONS

The present application claims the right of priority of European patent application EP20215495 filed with the European Patent Office on 18 Dec. 2020, the entire content of which is incorporated herein for all purposes.

### SEQUENCE LISTING

This application contains a Sequence Listing in a computer readable form, which is incorporated herein by reference.

### TECHNICAL FIELD

The present invention relates to novel polypeptides capable of detoxifying Ochratoxin A (OTA) and methods (e.g., for detoxifying OTA) based thereon. The present invention further relates to compositions, kits, transgenic plants, transgenic seeds, transgenic pollen grains, transgenic host cells, transgenic spores, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof comprising one or more polypeptides of the present invention capable of detoxifying Ochratoxin A (OTA).

### BACKGROUND OF THE INVENTION

Mycotoxins are secondary low molecular-weight metabolites produced by fungal species mainly belonging to Aspergillus and Penicillium genera. The mycotoxin ochratoxin A (OTA; also termed e.g. N-{[(3R)-5-chloro-8-hydroxy-3-methyl-1-oxo-3,4-dihydro-1H-2-benzopyran-7-yl]carbonyl}-L-phenylalanine, (−)-N-((5-chloro-8-hydroxy-3-methyl-1-oxo-7-isochromanyl)carbonyl)-3-phenylalanine, (25)-2-{[(3R)-5-chloro-8-hydroxy-3-methyl-1-oxo-3,4-dihydro-1H-2-benzopyran-7-carbonyl]amino}-3-phenylpropanoic acid, (R)-N-((5-chloro-3,4-dihydro-8-hydroxy-3-methyl-1-oxo-1H-2-benzopyran-7-yl)carbonyl)phenylalanine, N-(((3R)-5-chloro-8-hydroxy-3-methyl-1-oxo-7- isochromanyl)carbonyl)-3-phenyl-L-alanine, N-[(3R)-5-chloro-8-hydroxy-3-methyl-1-oxo-3,4-dihydro-1H-2-benzopyran-7-carbonyl]-L-phenylalanine, N-{[(3R)-5-chloro-8-hydroxy-3-methyl-1-oxo-3,4-dihydro-1H-isochromen-7-yl]carbonyl}-L-phenylalanine), CAS No. 303-47-9, is abundantly produced by fungi and the most toxic member of the ochratoxins group comprising inter alia ochratoxin B, ochratoxin C etc. In particular, OTA imposes serious concerns for food and feed safety due to severe adverse effects on humans and animals, including nephrotoxicity, immunotoxicity and carcinogenicity (Carballo et al., 2019; Malier et al., 2016). In this respect, OTA has been classified by the International Agency of Research on Cancer (IARC) in Group 2B as possible human carcinogen (IARC, 2012).

The environmental conditions of pre- and post-harvest stage of the edible crop, plant and related products influence the propagation of OTA-producing fungi, which lead to contamination with OTA and possibly other mycotoxins in food and feed materials. Notably, the presence of OTA has also been reported in cereal-based food and feed as well as in egg, meat, milk and products derived therefrom as a consequence of carry-over phenomena when animals were fed with contaminated feed. Further OTA presence was reported in a wide range of food items such as coffee, wine, beer, grapes, pulse, cocoa, etc. Due to the risk associated with OTA to the health and wellbeing of humans and animals, the European Commission has provided guidelines to control its presence in food (EC 1881/2006) and feed (EC 2016/1319). Therein, a tolerable weekly intake of 120 ng of OTA per kg of body weight was reported. With particular regards to animal feed, the guidance value for cereals/cereal products is 0.25 mg of OTA per kg of feed, and 0.05 mg/kg, 0.1 mg/kg and 0.01 mg/kg for compound feed for pigs, poultry and cats/dogs, respectively.

In general, a reduction of the mycotoxin content, in particular the OTA content, in food and feed materials is an unmet need of the food and feed industry striving to ensure a safe supply chain. Reports are available on the microbial detoxification of OTA using whole cells of Phenylobacterium immobile or Trichosporon mycotoxinivorans for OTA hydrolysis (Wegst and Lingens 1983; US 2009/0098244 A1). However, the applicability of microbes for OTA hydrolysis in food and feed applications is challenging due to regulatory and safety requirements. Additionally, the use of whole cell microbes is associated with complex and expensive requirements for cultivating and formulating the microbes in a manner ensuring sufficient viability and thus activity by said microbes.

As an alternative to microbial detoxification of OTA, it would be desirable to develop an enzyme-based strategy for biochemical OTA degradation. In this regard, an enzyme termed ochratoxinase from Aspergillus niger was previously reported to possess OTA hydrolyzing activity (e.g., Dobritzsch et al., 2014, Biochem J. 15; 462(3):441-52). However, an application of this enzyme under real-life conditions as required in the food and feed production line has not been shown and is considered unlikely. Dellafiora et al. (Toxins 2020, 12, 258) reported a porcine carboxypeptidase B as OTA hydrolyzing enzyme. EP 2 613 647 B1 discloses an amidase capable of degrading OTA. Notwithstanding, the OTA hydrolyzing enzymes reported so far cannot be expected to effectively perform in the application conditions due to insufficient activity, stability and/or recombinant reducibility.

Accordingly, there is a need to provide further improved OTA-hydrolyzing enzymes with improved functionality (e.g., enzymatic kinetics and/or efficiency) and/or stability (e.g., thermostability).

### SUMMARY OF THE INVENTION

The present invention relates to a polypeptide capable of detoxifying Ochratoxin A (OTA) and/or at least one OTA derivative, wherein said polypeptide is one or more of the following: (a) a polypeptide having at least 60% sequence identity (e.g. at least 64%, 70%, 74%, 80%, 90%, 91%, 92%, 93%, 94%, 95%, 96%, 97%, 98%, 99%) to an amino acid sequence selected from the group consisting of: SEQ ID NO: 1, SEQ ID NO: 2, SEQ ID NO: 3 and SEQ ID NO: 4; (b) a variant of the polypeptide (a), wherein said variant comprising a substitution, deletion, and/or insertion at one or more positions; (c) a fragment of the polypeptide of (a) or (b), wherein said fragment is capable of detoxifying OTA and/or at least one OTA derivative.

The present application satisfies this need by the provision of polypeptides described herein below, characterized in the claims and illustrated by the appended Examples and Figures.

### Overview of the Sequence Listing

As described herein references can be made to UniProtKB Accession Numbers (http://www.uniprot.org/, e.g., as available in UniProtKB release 2020_06 published Dec. 2, 2020).

SEQ ID NO: 1 is the amino acid sequence of the OTA-hydrolyzing polypeptide 1 of the present invention.

SEQ ID NO: 2 is the amino acid sequence of the OTA-hydrolyzing polypeptide 2 of the present invention.

SEQ ID NO: 3 is the amino acid sequence of the OTA-hydrolyzing polypeptide 3 of the present invention.

SEQ ID NO: 4 is the amino acid sequence of the OTA-hydrolyzing polypeptide 4 of the present invention. SEQ ID NOs: 5-23 are exemplary amino acid motifs of the present invention.

### BRIEF DESCRIPTION OF THE DRAWINGS

FIG. 1: Exemplary scheme of Ochratoxin A hydrolysis into phenylalanine and Ochratoxin a (Dellafiora et al., Toxins 2020, 12, 258).

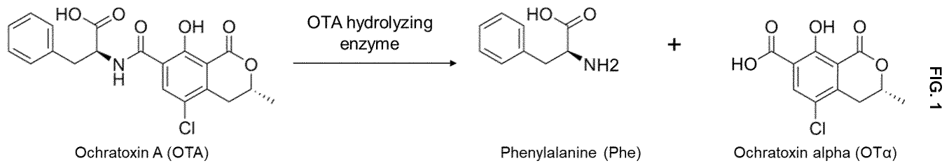

### DETAILED DESCRIPTION OF THE INVENTION

### Definitions

As referred herein “EC numbers” (Enzyme Commission numbers) may be used to refer to enzymatic activity according to the Enzyme nomenclature database, Release of February 26, 2020 (e.g., available at https://enzyme.expasy.org/). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide(s)” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is affected e.g. by post-translational modifications like glycosylation, acetylation, phosphorylation and the like. Such modifications are well known in the art.

Amino acid motif: The term “amino acid motif” or “the motif” as used herein may refer to a specifically defined amino acid stretch of a polypeptide. Thus, an amino acid motif of the prevent invention (e.g., as shown in SEQ ID NOs: 5-23) may relate to a short sequence of amino acids within a polypeptide (e.g., in SEQ ID NOs: 1-4).

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the no-brief option) is used as the percent identity and is calculated as follows: (Identical Residuesx100)/(Length of Alignment-Total Number of Gaps in Alignment).

Alternatively, the parameters used may be gap open penalty of 10, gap extension penalty of 0.5, and the EDNAFULL (EMBOSS version of NCBI NUC4.4) substitution matrix. The output of Needle labeled “longest identity” (obtained using the no-brief option) is used as the percent identity and is calculated as follows:

(Identical Deoxyribonucleotidesx100)/(Length of Alignment−Total Number of Gaps in Alignment).

Expression: The term “expression” includes any step involved in the production of a variant (polypeptide) including, but not limited to, transcription, post-transcriptional modification, translation, post-translational modification, and secretion.

Expression vector: The term “expression vector” may refer to a linear or circular DNA molecule that comprises a polynucleotide encoding a variant (polypeptide) and is operably linked to control sequences that provide for its expression, in particular for its transcription.

Fragment: The term “fragment” may refer to a polypeptide having one or more (e.g. several, e.g., 1, 2, 3, 4, 5, 6, 7, 8, 9 or 10, up to 15 or up to 20, etc.) amino acids absent from the amino and/or carboxyl terminus of a mature polypeptide; wherein the fragment has an activity as described elsewhere herein.

Host cell: The term “host cell” may refer to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication, e.g., recombinant or transgenic host cell (e.g., as in Example 1 herein).

Nucleic acid construct: The term “nucleic acid construct” may refer to a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” may refer to a configuration in which a control sequence is placed at an appropriate position relative to the coding sequence of a polynucleotide such that the control sequence directs expression of the coding sequence.

Control sequences: The term “control sequences” as used herein may refer to nucleic acid sequences necessary for expression of a polynucleotide encoding a variant (polynucleotide) of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide of the present invention.

As used herein, the term “corresponding to” may refer to a way of determining the specific amino acid of a sequence wherein reference is made to a specific amino acid sequence (e.g., US2020071638). E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NOs: 1, 2, 3 or 4 or any other sequence listed herein, has been described elsewhere herein. Alternative alignment methods may be used, and are well-known for the skilled person.

The term “position” when used in accordance with the present invention may refer to a position of an amino acid within an amino acid sequence depicted herein. The term “corresponding” in this context may include that a position is not only determined by the number of the preceding nucleotides/amino acids.

As used herein, “silent” mutations mean base substitutions within a nucleic acid sequence which do not change the amino acid sequence encoded by the nucleic acid sequence. “Conservative or equivalent” substitutions (or mutations) mean substitutions as listed as “Exemplary Substitutions” in Table I below. “Highly conservative” substitutions as used herein mean substitutions as shown under the heading “Preferred Substitutions” in Table I below.

TABLE I
Amino Acid Substitutions

| | | Preferred |
|---|---|---|
| Original | Exemplary Substitutions | Substitutions |
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; | ile |
| | ala; | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

Variant: The term “variant” may refer to a polypeptide having specific activity as described herein comprising an alteration, i.e., a substitution, insertion, and/or deletion, at one or more (e.g., several, e.g., 1, 2, 3, 4, 5, 6, 7, 8, 9 or 10, up to 15 or up to 20, etc.) positions. A substitution means replacement of the amino acid occupying a position with a different amino acid; a deletion means removal of the amino acid occupying a position; and an insertion means adding an amino acid adjacent to and immediately following the amino acid occupying a position.

As used herein the term “transgenic” may refer to an organism whose genome has been altered by the incorporation of foreign genetic material or additional copies of native genetic material, e.g. by transformation or recombination (e.g., U.S. Pat. No. 7,410,800B2). The transgenic organism may be a plant, mammal, fungus, bacterium or virus. As used herein “transgenic plant, seed or pollen grain” may refer to a plant, seed or pollen grain or progeny plant, seed or pollen grain of any subsequent generation derived therefrom, wherein the DNA of the plant, seed or pollen grain or progeny thereof contains an introduced exogenous DNA not originally present in a non-transgenic plant, seed or pollen grain of the same strain. The transgenic plant, seed or pollen grain may additionally contain sequences which are native to the plant being transformed, but wherein the exogenous DNA has been altered in order to alter the level or pattern of expression of the coding sequence.

The term “foodstuff” may refer to a substance having a food value.

The term “fodder” may refer to a substance fed to domestic animals.

The term “feed” may refer to a substance used as food for livestock.

The term “additive” may refer to a compound or substance added to another product or substance, e.g., in a small amount, to affect a desired property and/or characteristics.

The term “prebiotic” may refer to a compound or substance capable of inducing the growth and/or activity of beneficial microorganisms.

The term “detoxifying agent” may refer to a compound or substance capable of reducing- and/or inhibiting toxicity.

The term “nutritional supplement” may refer to a compound or substance capable to support the nutritional content of the diet, e.g., vitamins and minerals.

The term “phycophytic substance” may refer to a substance derived from a seaweed or algae species.

The term “intermediate” may refer to a compound or substance produced during the process (e.g., during an intermediate stage of the process) of obtaining an end-product of the present invention, e.g., foodstuff, fodder, fodder; feed, additive (e.g., foodstuff-, fodder- or feed additive), detoxifying agent, nutritional supplement or prebiotic of the present invention.

The term “OTA derivative” as used herein may refer to a compound or substance selected from the group consisting of: Furylacryloylphenylalanine (also called e.g. (2S)-2-[3-(furan-2-yl)prop-2-enoylamino]-3-phenylpropanoic acid), N-(3-(2-Furyl)acryloyl)phenylalanine (also called e.g. (2S)-2-[[(E)-3-(furan-2-yl)prop-2-enoyl]amino]-3-phenylpropanoic acid), L-Phenylalanine, N-(2-hydroxybenzoyl)- (also called e.g. (2S)-2-[(2-hydroxybenzoyl)amino]-3-phenylpropanoic acid), (2S)-2-[(4-Ethynylbenzoyl)amino]-3-phenylpropanoic acid (also called e.g. (2S)-2-[(4-ethynylbenzoyl)amino]-3-phenylpropanoic acid), Cinnamoyl-(trans)-L-phenylalanine (also called e.g. (2S)-3-phenyl-2-(3-phenylprop-2-enoylamino)propanoic acid), 2-(2-Phenoxy-acetylamino)-3-phenyl-propionic acid (also called e.g. 2-[(2-phenoxyacetyl)amino]-3-phenylpropanoic acid), N-(Phenoxyacetyl)phenylalanine (also called e.g. (2S)-2-[(2-phenoxyacetyl)amino]-3-phenylpropanoic acid), N-(2-Methoxybenzoyl)-L-phenylalanine (also called e.g. (2S)-2-[(2-methoxybenzoyl)amino]-3-phenylpropanoic acid), 3-Phenyl-2-[[(E)-3-thiophen-2-ylprop-2-enoyl]amino]propanoic acid (also called e.g. 3-phenyl-2-[[(E)-3-thiophen-2-ylprop-2-enoyl]amino]propanoic acid), N-[(E)-3-(2-Thienyl)acryloyl]-L-Phe-OH (also called e.g. (2S)-3-phenyl-2-[[(E)-3-thiophen-2-ylprop-2-enoyl]amino]propanoic acid), (2S)-3-Phenyl-2-(3-thiophen-2-ylprop-2-enoylamino)propanoic acid (also called e.g. (2S)-3-phenyl-2-(3-thiophen-2-ylprop-2-enoylamino)propanoic acid), 2-(1H-Indole-4-carbonylamino)-3-phenylpropanoic acid (also called e.g. 2-(1H-indole-4-carbonylamino)-3-phenylpropanoic acid), (2S)-2-(1H-Indole-4-carbonylamino)-3-phenylpropanoic acid (also called e.g. (2S)-2-(1H-indole-4-carbonylamino)-3-phenylpropanoic acid), 2-[(1-Benzofuran-2-yl)formamido]-3-phenylpropanoic acid (also called e.g. 2-(1-benzofuran-2-carbonylamino)-3-phenylpropanoic acid), (2S)-2-(2,3-Dihydro-1H-indene-5-carbonylamino)-3-phenylpropanoic acid (also called e.g. (2S)-2-(2,3-dihydro-1H-indene-5-carbonylamino)-3-phenylpropanoic acid), L-Phenylalanine, N-[3-(4-methylphenyl)-1-oxo-2-propenyl]- (also called e.g. (2S)-2-[3-(4-methylphenyl)prop-2-enoylamino]-3-phenylpropanoic acid), 2-(1,3-Dihydroisoindole-2-carbonylamino)-3-phenylpropanoic acid (also called e.g. 2-(1,3-dihydroisoindole-2-carbonylamino)-3-phenylpropanoic acid), (2S)-2-[3-(4-Hydroxyphenyl)prop-2-enoylamino]-3-phenylpropanoic acid (also called e.g. (2S)-2-[3-(4-hydroxyphenyl)prop-2-enoylamino]-3-phenylpropanoic acid), N-[(4-Methylphenoxy)acetyl]phenylalanine (also called e.g. 2-[[2-(4-methylphenoxy)acetyl]amino]-3-phenylpropanoic acid), L-Phenylalanine, N-[3-(4-fluorophenyl)-1-oxo-2-propenyl]- (also called e.g. (2S)-2-[3-(4-fluorophenyl)prop-2-enoylamino]-3-phenylpropanoic acid), 3-Phenyl-2-[(3,4,5-trihydroxybenzoyl)amino]propanoic acid (also called e.g. 3-phenyl-2-[(3,4,5-trihydroxybenzoyl)amino]propanoic acid), (2S)-3-Phenyl-2-[(3,4, 5-trihydroxybenzoyl)amino]propanoic acid (also called e.g. (2S)-3-phenyl-2-[(3,4,5-trihydroxybenzoyl)amino]propanoic acid), (2S)-2-[[3-(Chloromethyl)benzoyl]amino]-3-phenylpropanoic acid (also called e.g. (2S)-2-[[3-(chloromethyl)benzoyl]amino]-3-phenylpropanoic acid), (2S)-2-[(5-Methyl-1H-indole-2-carbonyl)amino]-3-phenylpropanoic acid (also called e.g. (2S)-2-[(5-methyl-1H-indole-2-carbonyl)amino]-3-phenylpropanoic acid), N-[(4-Butylphenyl)carbonyl]phenylalanine (also called e.g. 2-[(4-butylbenzoyl)amino]-3-phenylpropanoic acid), (2S)-3-(4-Hydroxyphenyl)-2-[3-(4-methylphenyl)prop-2-enoylamino]propanoic acid (also called e.g. (2S)-3-(4-hydroxyphenyl)-2-[3-(4-methylphenyl)prop-2-enoylamino]propanoic acid), N-(4-Hydroxycinnamoyl)tyrosine (also called e.g. 3-(4-hydroxyphenyl)-2-[[(E)-3-(4-hydroxyphenyl)prop-2-enoyl]amino]propanoic acid), N-[3-(4-Hydroxyphenyl)acryloyl]-L-tyrosine (also called e.g. (2S)-3-(4-hydroxyphenyl)-2-[3-(4-hydroxyphenyl)prop-2-enoylamino]propanoic acid), (2S)-3-(4-Hydroxyphenyl)-2-[(2-hydroxy-3-phenylprop-2-enoyl)amino]propanoic acid (also called e.g. (2S)-3-(4-hydroxyphenyl)-2-[(2-hydroxy-3-phenylprop-2-enoyl)amino]propanoic acid), Alanine, N-((5-chloro-8-hydroxy-3-methyl-1-oxo-7-isochromanyl)carbonyl)- (also called e.g. (2S)-2-[[(3R)-5-chloro-8-hydroxy-3-methyl-1-oxo-3,4-dihydroisochromene-7-carbonyl]amino]propanoic acid), L-Phenylalanine, N-[3-(4-chlorophenyl)-1-oxo-2-propenyl]- (also called e.g. (2S)-2-[3-(4-chlorophenyl)prop-2-enoylamino]-3-phenylpropanoic acid), 2-[[4-(2-Fluoroethoxy)benzoyl]amino]-3-phenylpropanoic acid (also called e.g. 2-[[4-(2-fluoroethoxy)benzoyl]amino]-3-phenylpropanoic acid), (2S)-2-[[2-(4-Chlorophenoxy)aetyl]amino]-3-phenylpropanoic acid (also called e.g. (2S)-2-[[2-(4-chlorophenoxy)acetyl]amino]-3-phenylpropanoic acid), (2S)-3-Phenyl-2-[[4-(trifluoromethyl)benzoyl]amino]propanoic Acid (also called e.g. (2S)-3-phenyl-2-[[4-(trifluoromethyl)benzoyl]amino]propanoic acid), Methyl (2S)-3-phenyl-2-[[(2S)-1,2,3,4-tetrahydronaphthalene-2-carbonyl]amino]propanoate (also called e.g. methyl (2S)-3-phenyl-2-[[(2S)-1,2,3,4-tetrahydronaphthalene-2-carbonyl]amino]propanoate), 2-[(5-Methoxy-1-benzofuran-2-carbonyl)amino]-3-pyridin-2-ylpropanoic acid (also called e.g. 2-[(5-methoxy-1-benzofuran-2-carbonyl)amino]-3-pyridin-2-ylpropanoic acid), (2S)-2-[(5-Methoxy-1-benzofuran-2-carbonyl)amino]-3-pyridin-2-ylpropanoic acid (also called e.g. (2S)-2-[(5-methoxy-1-benzofuran-2-carbonyl)amino]-3-pyridin-2-ylpropanoic acid), 2-[(5-Methoxy-1-benzofuran-2-carbonyl)amino]-3-pyridin-3-ylpropanoic acid (also called e.g. 2-[(5-methoxy-1-benzofuran-2-carbonyl)amino]-3-pyridin-3-ylpropanoic acid), (2S)-2-[(5-Methoxy-1-benzofuran-2-carbonyl)amino]-3-pyridin-3-ylpropanoic acid (also called e.g. (2S)-2-[(5-methoxy-1-benzofuran-2-carbonyl)amino]-3-pyridin-3-ylpropanoic acid), 2-[(5-Chloro-1H-indole-2-carbonyl)-amino]-3-phenyl-propionic acid (also called e.g. 2-[(5-chloro-1H-indole-2-carbonyl)amino]-3-phenylpropanoic acid), 2-[(5-Fluoro-1-benzothiophen-2-yl)formamido]-3-phenylpropanoic acid (also called e.g. 2-[(5-fluoro-1-benzothiophene-2-carbonyl)amino]-3-phenylpropanoic acid), (S)-2-(5-Fluorobenzo[b]thiophene-2-carboxamido)-3-phenylpropanoic acid (also called e.g. (2S)-2-[(5-fluoro-1-benzothiophene-2-carbonyl)amino]-3-phenylpropanoic acid), 2-[(6-Fluoro-1-benzothiophene-2-carbonyl)amino]-3-phenylpropanoic acid (also called e.g. 2-[(6-fluoro-1-benzothiophene-2-carbonyl)amino]-3-phenylpropanoic acid), N-((5-Chloro-8-hydroxy-3-methyl-1-oxo-7-isochromanyl)carbonyl)serine (also called e.g. (2S)-2-[[(3R)-5-chloro-8-hydroxy-3-methyl-1-oxo-3,4-dihydroisochromene-7-carbonyl]amino]-3-hydroxypropanoic acid), 2-(S)-[(5-Chloro-1H-pyrrolo[2,3-c]pyridine-2-carbonyl)amino]-3-phenylpropionic acid (also called e.g. (2S)-2-[(5-chloro-1H-pyrrolo[2,3-c]pyridine-2-carbonyl)amino]-3-phenylpropanoic acid), (2S)-2-[[(Z)-3-Chloro-3-(4-methylphenyl)prop-2-enoyl]amino]-3-phenylpropanoic acid (also called e.g. (2S)-2-[[(Z)-3-chloro-3-(4-methylphenyl)prop-2-enoyl]amino]-3-phenylpropanoic acid), (2S)-3-Phenyl-2-[(4-phenylbenzoyl)amino]propanoic acid (also called e.g. (2S)-3-phenyl-2-[(4-phenylbenzoyl)amino]propanoic acid), (2S)-3-(3-Chlorophenyl)-2-[[5-(dimethylamino)pyridine-2-carbonyl]amino]propanoic acid (also called e.g. (2S)-3-(3-chlorophenyl)-2-[[5-(dimethylamino)pyridine-2-carbonyl]amino]propanoic acid), N-[(5-Methoxy-1-methyl-1H-indo1-2-yl)carbonyl]-L-phenylalanine (also called e.g. (2S)-2-[(5-methoxy-1-methylindole-2-carbonyl)amino]-3-phenylpropanoic acid), (2S)-2-[(2-Methyl-1-oxo-3,4-dihydroisoquinoline-3-carbonyl)amino]-3-phenylpropanoic acid (also called e.g. (2S)-2-[(2-methyl-1-oxo-3,4-dihydroisoquinoline-3-carbonyl)amino]-3-phenylpropanoic acid), Proline, 1-((5-chloro-8-hydroxy-3-methyl-1-oxo-7-isochromanyl)carbonyl)- (also called e.g. (2S)-1-[(3R)-5-chloro-8-hydroxy-3-methyl-1-oxo-3,4-dihydroisochromene-7-carbonyl]pyrrolidine-2-carboxylic acid), 3-(3-Fluorophenyl)-2-[[4-(trifluoromethyl)benzoyl]amino]propanoic acid (also called e.g. 3-(3-fluorophenyl)-2-[[4-(trifluoromethyl)benzoyl]amino]propanoic acid), N-((5-Chloro-8-hydroxy-3-methyl-1-oxo-7-isochromanyl)carbonyl)valine (also called e.g. (2S)-2-[[(3R)-5-chloro-8-hydroxy-3-methyl-1-oxo-3,4-dihydroisochromene-7-carbonyl]amino]-3-methylbutanoic acid), N-[(5-Chloro-1-methyl-1H-indo1-2-yl)carbonyl]-L-phenylalanine (also called e.g. (2S)-2-[(5-chloro-1-methylindole-2-carbonyl)amino]-3-phenylpropanoic acid), 3-(2-Fluorophenyl)-2-[(5-methoxy-1-benzofuran-2-carbonyl)amino]propanoic acid (also called e.g. 3-(2-fluorophenyl)-2-[(5-methoxy-1-benzofuran-2-carbonyl)amino]propanoic acid), (2S)-3-(2-Fluorophenyl)-2-[(5-methoxy-1-benzofuran-2-carbonyl)amino]propanoic acid (also called e.g. (2S)-3-(2-fluorophenyl)-2-[(5-methoxy-1-benzofuran-2-carbonyl)amino]propanoic acid), N-((5-Chloro-8-hydroxy-3-methyl-1-oxo-7-isochromanyl)carbonyl)threonine (also called e.g. (2S,3R)-2-[[(3R)-5-chloro-8-hydroxy-3-methyl-1-oxo-3,4-dihydroisochromene-7-carbonyl]amino]-3-hydroxybutanoic acid), (2S)-2-[(2,3,4,5,6-Pentafluorobenzoyl)amino]-3-phenylpropanoic acid (also called e.g. (2S)-2-[(2,3,4,5,6-pentafluorobenzoyl)amino]-3-phenylpropanoic acid), N-((5-Chloro-8-hydroxy-3-methyl-1-oxo-7-isochromanyl)carbonyl)cysteine (also called e.g. 2-[(5-chloro-8-hydroxy-3-methyl-1-oxo-3,4-dihydroisochromene-7-carbonyl)amino]-3-sulfanylpropanoic acid), (2S)-2-[(3-Chloro-4-prop-2-enoxybenzoyl)amino]-3-phenylpropanoic acid (also called e.g. (2S)-2-[(3-chloro-4-prop-2-enoxybenzoyl)amino]-3-phenylpropanoic acid), 2-[(6-Methyl-5,8-dioxonaphthalene-2-carbonyl)amino]-3-phenylpropanoic acid (also called e.g. 2-[(6-methyl-5,8-dioxonaphthalene-2-carbonyl)amino]-3-phenylpropanoic acid), (S)-2-[(2-Butyl-4-chloro-1-methyl-1H-imidazole-5-yl)carbonylamino]-3-phenylpropionic acid (also called e.g. (2S)-2-[(2-butyl-5-chloro-3-methylimidazole-4-carbonyl)amino]-3-phenylpropanoic acid), Ochratoxin B (also called e.g. (2S)-2-[[(3R)-8-hydroxy-3-methyl-1-oxo-3,4-dihydroisochromene-7-carbonyl]amino]-3-phenylpropanoic acid), 2-[(8-Hydroxy-3-methyl-1-oxoisochroman-7-carbonyl)amino]-3-phenylpropionic acid (also called e.g. 2-[(8-hydroxy-3-methyl-1-oxo-3,4-dihydroisochromene-7-carbonyl)amino]-3-phenylpropanoic acid), 2-[(8-Hydroxy-5-methyl-1-oxo-3,4-dihydroisochromene-7-carbonyl)amino]-3-phenylpropanoic acid (also called e.g. 2-[(8-hydroxy-5-methyl-1-oxo-3,4-dihydroisochromene-7-carbonyl)amino]-3-phenylpropanoic acid), 1-((5-Chloro-8-hydroxy-3-methyl-1-oxo-7-isochromanyl)carbonyl)-4-hydroxyproline (also called e.g. 1-(5-chloro-8-hydroxy-3-methyl-1-oxo-3,4-dihydroisochromene-7-carbonyl)-4-hydroxypyrrolidine-2-carboxylic acid), Leucine, N-((5-chloro-8-hydroxy-3-methyl-1-oxo-7-isochromanyl)carbonyl)- (also called e.g. (2S)-2-[[(3R)-5-chloro-8-hydroxy-3-methyl-1-oxo-3,4-dihydroisochromene-7-carbonyl]amino]-4-methylpentanoic acid), 2-[(1-Chloro-4-hydroxyisoquinoline-3-carbonyl)amino]-3-phenylpropanoic acid (also called e.g. 2-[(1-chloro-4-hydroxyisoquinoline-3-carbonyl)amino]-3-phenylpropanoic acid), 3-(4-Chlorophenyl)-2-[[4-(trifluoromethyl)benzoyl]amino]propanoic acid (also called e.g. 3-(4-chlorophenyl)-2-[[4-(trifluoromethyl)benzoyl]amino]propanoic acid), Aspartic acid, N4(5-chloro-8-hydroxy-3-methyl-1-oxo-7-isochromanyl)carbonyl)- (also called e.g. (2S)-2-[[(3R)-5-chloro-8-hydroxy-3-methyl-1-oxo-3,4-dihydroisochromene-7-carbonyl]amino]butanedioic acid), 2-(5H-Chromeno[3,4-c]pyridine-8-carbonylamino)-3-phenylpropanoic acid (also called e.g. 2-(5H-chromeno[3,4-c]pyridine-8-carbonylamino)-3-phenylpropanoic acid), (S)-2-(3-Chloro-5-fluorobenzo[b]thiophene-2-carboxamido)-3-phenylpropanoic acid (also called e.g. (2S)-2-[(3-chloro-5-fluoro-1-benzothiophene-2-carbonyl)amino]-3-phenylpropanoic acid), N-[(4,7-Dimethoxy-1-methyl-1 H-indol-2-yl)carbonyl]-L-phenylalanine (also called e.g. (2S)-2-[(4,7-dimethoxy-1-methylindole-2-carbonyl)amino]-3-phenylpropanoic acid), Ochratoxin B methyl ester (also called e.g. methyl (2S)-2-[[(3R)-8-hydroxy-3-methyl-1-oxo-3,4-dihydroisochromene-7-carbonyl]amino]-3-phenylpropanoate), L-Phenylalanine, N4(3,4-dihydro-4,8-dihydroxy-3-methyl-1-oxo-1H-2-benzopyran-7-yl)carbonyl)-, (3R-trans)- (also called e.g. (2S)-2-[[(3R,4S)-4,8-dihydroxy-3-methyl-1-oxo-3,4-dihydroisochromene-7-carbonyl]amino]-3-phenylpropanoic acid), 4-Hydroxyochratoxin B (also called e.g. (2S)-2-[[(3R)-4,8-dihydroxy-3-methyl-1-oxo-3,4-dihydroisochromene-7-carbonyl]amino]-3-phenylpropanoic acid), 4-Hydroxyochratoxin B, (4R)- (also called e.g. (2S)-2-[[(3R,4R)-4,8-dihydroxy-3-methyl-1-oxo-3,4-dihydroisochromene-7-carbonyl]amino]-3-phenylpropanoic acid), Ochratoxin hydroquinone (also called e.g. (2S)-2-[[(3R)-5,8-dihydroxy-3-methyl-1-oxo-3,4-dihydroisochromene-7-carbonyl]amino]-3-phenylpropanoic acid), Glutamic acid, N4(5-chloro-8-hydroxy-3-methyl-1-oxo-7-isochromanyl)carbonyl)- (also called e.g. (2S)-2-[[(3R)-5-chloro-8-hydroxy-3-methyl-1-oxo-3,4-dihydroisochromene-7-carbonyl]amino]pentanedioic acid), 2-[(1-Chloro-4-hydroxyisoquinoline-3-carbonyl)amino]-3-(4-hydroxyphenyl)propanoic acid (also called e.g. 2-[(1-chloro-4-hydroxyisoquinoline-3-carbonyl)amino]-3-(4-hydroxyphenyl)propanoic acid), N-((5-Chloro-8-hydroxy-3-methyl-1-oxo-7- isochromanyl)carbonyl)methionine (also called e.g. (2S)-2-[[(3R)-5-chloro-8-hydroxy-3-methyl-1-oxo-3,4-dihydroisochromene-7-carbonyl]amino]-4-methylsulfanylbutanoic acid), 2-[[(3R)-5-Chloro-8-hydroxy-3-methyl-3,4-dihydro-1H-isochromene-7-carbonyl]amino]-3-phenylpropanoic acid (also called e.g. 2-[[(3R)-5-chloro-8-hydroxy-3-methyl-3,4-dihydro-1H-isochromene-7-carbonyl]amino]-3-phenylpropanoic acid), (2S)-2-[[(3R)-5-Chloro-8-hydroxy-3-methyl-3,4-dihydro-1H-isochromene-7-carbonyl]amino]-3-phenylpropanoic acid (also called e.g. (2S)-2-[[(3R)-5-chloro-8-hydroxy-3-methyl-3,4-dihydro-1H-isochromene-7-carbonyl]amino]-3-phenylpropanoic acid), (2S)-2-[(5-Chloro-8-hydroxy-1-oxo-3,4-dihydroisochromene-7-carbonyl)amino]-3-phenylpropanoic acid (also called e.g. (2S)-2-[(5-chloro-8-hydroxy-1-oxo-3,4-dihydroisochromene-7-carbonyl)amino]-3-phenylpropanoic acid), (2S)-2-[(4-lodobenzoyl)amino]-3-phenylpropanoic acid (also called e.g. (2S)-2-[(4-iodobenzoyl)amino]-3-phenylpropanoic acid), Ochratoxin B ethyl ester (also called e.g. ethyl 2-[(8-hydroxy-3-methyl-1-oxo-3,4-dihydroisochromene-7-carbonyl)amino]-3-phenylpropanoate), (R)N-(5-Chloro-3,4-dihydro-8-hydroxy-3-methyl-1-oxo-1H-2-benzopyran-7-yl)phenylalanine (also called e.g. 2-[(5-chloro-8-hydroxy-3-methyl-1-oxo-3,4-dihydroisochromene-7-carbonyl)amino]-3-phenylpropanoic acid), Phenylalanine-ochratoxin A (also called e.g. 2-[[(3R)-5-chloro-8-hydroxy-3-methyl-1-oxo-3,4-di hydroisochromene-7-carbonyl]amino]-3-phenylpropanoic acid), N-[(3R)-5-Chloro-8- hydroxy-3-methyl-1-oxo-3,4-dihydro-1H-2-benzopyran-7-carbonyl]-D-phenylalanine (also called e.g. (2R)-2-[[(3R)-5-chloro-8-hydroxy-3-methyl-1-oxo-3,4-dihydroisochromene-7-carbonyl]amino]-3-phenylpropanoic acid), 3S14S-Ochratoxin A (also called e.g. (2S)-2-[[(3S)-5-chloro-8-hydroxy-3-methyl-1-oxo-3,4-dihydroisochromene-7-carbonyl]amino]-3- phenylpropanoic acid), (R)-N-((5-Chloro-3,4-dihydro-8-hydroxy-3-methyl-1-oxo-1H-benzo(c)pyran-7-yl)carbonyl)-3-phenylalanine (also called e.g. (2R)-2-[(5-chloro-8-hydroxy-3-methyl-1-oxo-3,4-dihydroisochromene-7-carbonyl)amino]-3-phenylpropanoic acid), N-[(5-Chloro-3,4-dihydro-8-hydroxy-3-methyl-1-oxo-1H-2-benzopyran-7-yl)carbonyl]-L-phenylalanine (also called e.g. (2S)-2-[(5-chloro-8-hydroxy-3-methyl-1-oxo-3,4-dihydroisochromene-7-carbonyl)amino]-3-phenylpropanoic acid), 3S14R-Ochratoxin A (also called e.g. (2R)-2-[[(3S)-5-chloro-8-hydroxy-3-methyl-1-oxo-3,4-dihydroisochromene-7-carbonyl]amino]-3- phenylpropanoic acid), L-Tyrosine, N4(5-chloro-3,4-dihydro-3-methyl-1-oxo-1H-2-benzopyran-7-yl)carbonyl)- (also called e.g. (2S)-2-[(5-chloro-3-methyl-1-oxo-3,4-dihydroisochromene-7-carbonyl)amino]-3-(4-hydroxyphenyl)propanoic acid), 2-[(8-Hydroxy-5-methylchloranuidyl-1-oxo-3,4-dihydroisochromene-7-carbonyl)amino]-3-phenylpropanoic acid (also called e.g. 2-[(8-hydroxy-5-methylchloranuidyl-1-oxo-3,4-dihydroisochromene-7-carbonyl)amino]-3-phenylpropanoic acid), Ochratoxin A-D4 (also called e.g. (2S)-2-[[(3R)-5-chloro-3-deuterio-8-hydroxy-1-oxo-3-(trideuteriomethyl)-4H-isochromene-7-carbonyl]amino]-3- phenylpropanoic acid), Ochratoxin A-d5 solution, 10 mug/mL in acetonitrile, analytical standard (also called e.g. (2S)-2-[[(3R)-5-chloro-8-hydroxy-3-methyl-1-oxo-3,4-dihydroisochromene-7-carbonyl]amino]-3-(2,3,4,5,6-pentadeuteriophenyl)propanoic acid), (S)-3-(4-Bromophenyl)-2-(5-(tert-butyl)thiophene-2-carboxamido)propanoic acid (also called e.g. (2S)-3-(4-bromophenyl)-2-[(5-tert-butylthiophene-2-carbonyl)amino]propanoic acid), BrC1═CC═C(C═C1)CC(C(═O)O)NC(═O)C═1SC(═CC═1)C(C)(C)C (also called e.g. 3-(4-bromophenyl)-2-[(5-tert-butylthiophene-2-carbonyl)amino]propanoic acid), 5-Chloro-8-hydroxy-3-methyl-N-[2S)-1-(methylamino)-1-oxo-3-phenylpropan-2-yl]-1-oxo-3,4-dihydroisochromene-7-carboxamide (also called e.g. 5-chloro-8-hydroxy-3-methyl-N-[2S)-1-(methylamino)-1-oxo-3-phenylpropan-2-yl]-1-oxo-3,4-dihydroisochromene-7-carboxamide), Ochratoxin A methyl ester (also called e.g. methyl (2S)-2-[[(3R)-5-chloro-8-hydroxy-3-methyl-1-oxo-3,4-dihydroisochromene-7-carbonyl]amino]-3-phenylpropanoate), L-Phenylalanine, N-((5-chloro-3,4-dihydro-8-hydroxy-3-methyl-1,4-dioxo-1H-2-benzopyran-7-yl)carbonyl)-, (R)-(also called e.g. (2S)-2-[[(3R)-5-chloro-8-hydroxy-3-methyl-1,4-dioxoisochromene-7-carbonyl]amino]-3-phenylpropanoic acid), O-Methylochratoxin A (also called e.g. (2S)-2-[[(3R)-5-chloro-8-methoxy-3-methyl-1-oxo-3,4-dihydroisochromene-7-carbonyl]amino]-3-phenylpropanoic acid), (2R)-2-[(5-Chloro-8-hydroxy-3,3-dimethyl-1-oxo-4H-isochromene-7-carbonyl)amino]-3-phenylpropanoic acid (also called e.g. (2R)-2-[(5-chloro-8-hydroxy-3,3-dimethyl-1-oxo-4H-isochromene-7-carbonyl)amino]-3-phenylpropanoic acid), (2S)-2-[[(3R)-5-Chloro-3-ethyl-8-hydroxy-1-oxo-3,4-dihydroisochromene-7-carbonyl]amino]-3-phenylpropanoic acid (also called e.g. (2S)-2-[[(3R)-5-chloro-3-ethyl-8-hydroxy-1-oxo-3,4-dihydroisochromene-7-carbonyl]amino]-3-phenylpropanoic acid), 4-Hydroxyochratoxin A (also called e.g. (2S)-2-[[(3R,4 R)-5-chloro-4,8-dihydroxy-3-methyl-1-oxo-3,4-dihydroisochromene-7-carbonyl]amino]-3-phenylpropanoic acid), (2S)-2-[[(3R)-5-Chloro-8-hydroxy-3-(hydroxymethyl)-1-oxo-3,4-dihydroisochromene-7-carbonyl]amino]-3-phenylpropanoic acid (also called e.g. (2S)-2-[[(3R)-5-chloro-8-hydroxy-3-(hydroxymethyl)-1-oxo-3,4-dihydroisochromene-7-carbonyl]amino]-3-phenylpropanoic acid), (4S)-4-Hydroxyochratoxin A (also called e.g. (2S)-2-[[(3R,4S)-5-chloro-4,8-dihydroxy-3-methyl-1-oxo-3,4-dihydroisochromene-7-carbonyl]amino]-3-phenylpropanoic acid), N-((5-Chloro-8-hydroxy-3-methyl-1-oxo-7-isochromanyl)carbonyl)tyrosine (also called e.g. Ochratoxin TA, or (2S)-2-[(5-chloro-8-hydroxy-3-methyl-1-oxo-3,4-dihydroisochromene-7-carbonyl)amino]-3-(4-hydroxyphenyl)propanoic acid), 2-[[3,4-Dihydro-1-oxo-3-methyl-4,8-dihydroxy-5-chloro-1H-2-benzopyran-7-yl]carbonylamino]-3-phenylpropanoic acid (also called e.g. 2-[(5-chloro-4,8-dihydroxy-3-methyl-1-oxo-3,4-dihydroisochromene-7-carbonyl)amino]-3-phenylpropanoic acid), 10-Hydroxyochratoxin A (also called e.g. (2S)-2-[[(3S)-5-chloro-8-hydroxy-3-(hydroxymethyl)-1-oxo-3,4-dihydroisochromene-7-carbonyl]amino]-3- phenylpropanoic acid), 2-[(8-Hydroxy-5-methylchloranuidyl-1-oxo-3,4-dihydroisochromene-7-carbonyl)amino]-3-(4-hydroxyphenyl)propanoic acid (also called e.g. 2-[(8-hydroxy-5-methylchloranuidyl-1-oxo-3,4-dihydroisochromene-7-carbonyl)amino]-3-(4-hydroxyphenyl)propanoic acid), 3-[[(1S)-1-Carboxy-2-phenylethyl]carbamoyl]-5-chloro-2-hydroxy-6-[(2S)-2-hydroxypropyl]benzoic acid (also called e.g. 3-[[(1S)-1-carboxy-2-phenylethyl]carbamoyl]-5-chloro-2-hydroxy-6-[(2S)-2-hydroxypropyl]benzoic acid), Ochratoxin A 13C20 (also called e.g. (2S)-2-[[(3R)-5-chloro-8-hydroxy-3-(113C)methyl-1-oxo-3,4-dihydroisochromene-7-carbonyl]amino]-3- (1,2,3,4,5,6-13C6)cyclohexatrienyl)(1,2,3-13C3)propanoic acid), Ochratoxin C (also called e.g. ethyl (2S)-2-[[(3R)-5-chloro-8-hydroxy-3-methyl-1-oxo-3,4-di hydroisochromene-7-carbonyl]amino]-3-phenylpropanoate), Ochratoxin A-O-methyl, methyl ester (also called e.g. methyl (2S)-2-[[(3R)-5-chloro-8-methoxy-3-methyl-1-oxo-3,4-dihydroisochromene-7-carbonyl]amino]-3-phenylpropanoate), Ethyl 2-[(5-chloro-8-hydroxy-3-methyl-1-oxo-3,4-dihydro-1H-2-benzopyran-7-yl)formamido]-3-phenylpropanoate (also called e.g. ethyl 2-[(5-chloro-8-hydroxy-3-methyl-1-oxo-3,4-dihydroisochromene-7-carbonyl)amino]-3-phenylpropanoate), Ochratoxin TC (also called e.g. ethyl (2S)-2-[(5-chloro-8-hydroxy-3-methyl-1-oxo-3,4-dihydroisochromene-7-carbonyl)amino]-3-(4-hydroxyphenyl)propanoate), N-[[3-(4-Carboxybutyl)-5-chloro-3,4-dihydro-8-hydroxy-1-oxo-1 H-2-benzopyran-7-yl]carbonyl]-L-phenylalanine (also called e.g. 5-[7-[[(1S)-1-carboxy-2-phenylethyl]carbamoyl]-5-chloro-8-hydroxy-1-oxo-3,4-dihydroisochromen-3-yl]pentanoic acid), 6-({(2S)-2-[(5-Chloro-8-hydroxy-3-methyl-1-oxo-3,4-dihydro-1 H-2-benzopyran-7-carbonyl)amino]-3-phenylpropanoyl}amino)hexanoic acid (also called e.g. 6-[[(2S)-2-[(5-chloro-8-hydroxy-3-methyl-1-oxo-3,4-dihydroisochromene-7-carbonyl)amino]-3-phenylpropanoyl]amino]hexanoic acid), 4-(5-Carboxypentyl)-N-[(5-chloro-3,4-dihydro-8-hydroxy-3-methyl-1-oxo-1 H-2-benzopyran-7-yl)carbonyl]-L-phenylalanine (also called e.g. 6-[4-[(2S)-2-carboxy-2-[(5-chloro-8-hydroxy-3-methyl-1-oxo-3,4-dihydroisochromene-7-carbonyl)amino]ethyl]phenyl]hexanoic acid), 6-[4-[2-[(5-Chloro-8-hydroxy-3-methyl-1-oxo-3,4-dihydroisochromene-7-carbonyl)amino]-3-[(2-methylpropan-2-yl)oxy]-3-oxopropyl]phenyl]hexanoic acid (also called e.g. 6-[4-[2-[(5-chloro-8-hydroxy-3-methyl-1-oxo-3,4-dihydroisochromene-7-carbonyl)amino]-3-[(2-methylpropan-2-yl)oxy]-3- oxopropyl]phenyl]hexanoic acid), (2S)-2-[[3-[5-(1-Aminobutylamino)-5-oxopentyl]-5-chloro-8-hydroxy-1-oxo-3,4-dihydroisochromene-7-carbonyl]amino]-3-(3-propan-2-ylphenyl)propanoic acid (also called e.g. (2S)-2-[[3-[5-(1-aminobutylamino)-5-oxopentyl]-5-chloro-8-hydroxy-1-oxo-3,4-dihydroisochromene-7-carbonyl]amino]-3-(3-propan-2-ylphenyl)propanoic acid), Ochratoxin A cellobiose ester (also called e.g. [(2R,3S,4R,5R,6R)-4,5,6-trihydroxy-3-[(2S,3R,4S,5S,6R)-3,4,5-trihydroxy-6-(hydroxymethyl)oxan-2-yl]oxyoxan-2-yl]methyl (2S)-2-[[(3R)-5-chloro-8-hydroxy-3-methyl-1-oxo-3,4-dihydroisochromene-7-carbonyl]amino]-3-phenylpropanoate), Ochratoxin A phenol-glucuronide, Ochratoxin A amino-glucuronide, Ochratoxin A acyl-glucuronide. In particular, the term “OTA derivative” may refer to a compound or substance selected from the group consisting of ochratoxin B and ochratoxin C.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

The objective of the present invention has been achieved by providing means and methods as described herein, e.g., by providing polypeptide capable of detoxifying ochratoxin/s, e.g., Ochratoxin A (OTA) and/or at least one OTA derivative (e.g., as listed above, e.g., a compound or substance selected from the group consisting of: Furylacryloylphenylalanine (also called e.g. (2S)-2-[3-(furan-2-yl)prop-2-enoylamino]-3-phenylpropanoic acid), N-(3-(2-Furyl)acryloyl)phenylalanine (also called e.g. (2S)-2-[[(E)-3-(furan-2-yl)prop-2-enoyl]amino]-3-phenylpropanoic acid), L-Phenylalanine etc., preferably said at least one OTA derivative is ochratoxin B and/or ochratoxin C), wherein said polypeptide is a polypeptide having at least 64% sequence identity to the amino acid sequence selected from the group consisting of: SEQ ID NO: 1, SEQ ID NO: 2, SEQ ID NO: 3 and SEQ ID NO: 4. As shown in examples section below, a reduction of the OTA concentration in both, plasma and urine, was, for example, observed when polypeptide(s) of the present invention (e.g., SEQ ID NOs: 1-4) of the present invention was part of the diet setup, demonstrating the applicability of the enzymes of the present invention in food/feed for OTA detoxification.

In some embodiments/aspects, the present invention relates to a polypeptide capable of detoxifying (e.g., modifying or hydrolyzing) an ochratoxin (e.g., CN108120791), e.g., Ochratoxin A (OTA) and/or at least one OTA derivative (e.g., as defined herein, e.g., a compound or substance selected from the group consisting of: Furylacryloylphenylalanine (also called e.g. (25)-2-[3-(furan-2-Aprop-2-enoylamino]-3-phenylpropanoic acid), N-(3-(2-Furyl)acryloyl)phenylalanine (also called e.g. (25)-2-[[(E)-3-(furan-2-yl)prop-2-enoyl]amino]-3-phenylpropanoic acid), L-Phenylalanine etc., preferably said at least one OTA derivative is ochratoxin B and/or ochratoxin C), wherein said polypeptide is one or more of the following: (a) a polypeptide having at least 60% sequence identity (e.g. at least 64%, 65%, 66%, 67%, 68%, 69%, 70%, 71%, 72%, 73%, 74%, 80%, 81%, 82%, 83%, 84%, 85%, 86%, 87%, 88%, 89%, 90%, 91%, 92%, 93%, 94%, 95%, 96%, 97%, 98%, 99%) to an amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-4; further preferably said polypeptide having SEQ ID NO: 1; (b) a variant of the polypeptide (a), wherein said variant comprising a substitution, deletion, and/or insertion at one or more positions; (c) a fragment of the polypeptide of (a) or (b), wherein said fragment is capable of detoxifying OTA and/or at least one OTA derivative; (d) a polypeptide comprising one or more of the following amino acid sequences (e.g., conservative motifs): (i) GHACGHNLIA (SEQ ID NO: 5); (ii) IGSPAEEGGGGK (SEQ ID NO: 6); (iii) MAHP (SEQ ID NO: 7); (iv) VNHT (SEQ ID NO: 23).; (v) KVAKGLA (SEQ ID NO: 9); (vi) IGSPAEEGGGGKVIL (SEQ ID NO: 10); (vii) AHAGAAPWLG (SE ID NO: 11); (viii) PELGFHEVKA (SEQ ID NO: 12); (ix) TNPTSLPTAFVAT (SEQ ID NO: 13); (x) GGRTFGFNAEYDAL (SEQ ID NO: 14); (xi) GHACGHNLIAIVAVASA (SEQ ID NO: 15); (xii) IPGTIKVIGSPAEEGGGGKVILLNEG (SEQ ID NO: 16); (xiii) DACVMAHPEGG (SEQ ID NO: 17); (xiv) AHAGAAPWLGVNALDAAVQGY (SEQ ID NO: 18); (xv)

GHACGHNLIAXXAVAXAXGXKAAXXXXXIXGTXKXIGSPAEEGGGGKXILNEGXYD XXDACXMAHPXGG (SEQ ID NO: 19), wherein X is any amino acid; (xvi) (i), (ii), (iii) and (iv), e.g., SEQ ID NOs: 5, 6, 7 and 23; (xvii) AHAGAAPW (SEQ ID NO: 8).

In some embodiments/aspects of the present invention, the polypeptide is selected from the group consisting of: SEQ ID NOs: 1-4; preferably said polypeptide having SEQ ID NO: 1.

In some embodiments/aspects of the present invention, the polypeptide is capable of degrading ochratoxin A with a specific activity of at least 0.01 U/g (e.g. as shown in Example 2 herein, e.g., at least 0.05, 0.1, 0.5, 0.75, 1.0, 1.2, 2, 5, 7 or 10 U/g).

In some embodiments/aspects of the present invention, the polypeptide comprising one or more of the following amino acid sequences (e.g., conservative motifs): (i) GHACGHNLIA (SEQ ID NO: 5); (ii) IGSPAEEGGGGK (SEQ ID NO: 6); (iii) MAHP (SEQ ID NO: 7); (iv) VNHT (SEQ ID NO: 23); (v) KVAKGLA (SEQ ID NO: 9); (vi) IGSPAEEGGGGKVIL (SEQ ID NO: 10); (vii) AHAGAAPWLG (SE ID NO: 11); (viii) PELGFHEVKA (SEQ ID NO: 12); (ix) TNPTSLPTAFVAT (SEQ ID NO: 13); (x) GGRTFGFNAEYDAL (SEQ ID NO: 14); (xi) GHACGHNLIAIVAVASA (SEQ ID NO: 15); (xii) IPGTIKVIGSPAEEGGGGKVILLNEG (SEQ ID NO: 16); (xiii) DACVMAHPEGG (SEQ ID NO: 17); (xiv) AHAGAAPWLGVNALDAAVQGY (SEQ ID NO: 18); (xv)

GHACGHNLIAXXAVAXAXGXKAAXXXXXIXGTXKXIGSPAEEGGGGKXILXNEGXYD XXDACXMAHPXGG (SEQ ID NO: 19), wherein X is any amino acid; (xvi) (i), (ii), (iii) and (iv), e.g., SEQ ID NOs: 5, 6, 7, and 23; (xvii) AHAGAAPW (SEQ ID NO: 8); (xviii) a polypeptide having at least 70% sequence identity (e.g. at least 71%, 74%, 80%, 90%, 91%, 92%, 93%, 94%, 95%, 96%, 97%, 98%, 99%, 100%) to the amino acid sequence selected from the group consisting (i)-(xvii); (xix) a polypeptide having at least 70% identity to the amino acid sequence of (xv); and/or having at least 80% identity to any one of (viii)-(xiv); and/or having at least 90% to (vi) and/or (vii); (xx) a variant of the polypeptide having amino acid sequence (i), (ii), (iii), (iv), (v), (vi), (vii), (viii), (ix), (x), (xi), (xii), (xiii) or (xiv), wherein said variant comprises a substitution, deletion, and/or insertion at one or more positions; (xxi) a polypeptide comprising amino acid sequences: (i), (ii), (iii) and (iv).

In some embodiments/aspects of the present invention, said detoxifying Ochratoxin A (OTA) comprising or consisting of hydrolyzing an amide bond of OTA.

In some embodiments/aspects of the present invention, said polypeptide having amidohydrolase activity (e.g., EC 3.5.1.-, e.g., EC 3.5.1.14 (aminoacylase 1)). “EC 3.5.1.-” may refer to an EC number in which “-” can be any number from 1 to 135. For example, in this context “-” can be interchangeably used with “X”, i.e., EC 3.5.1.- can be interchangeably used with EC 3.5.1.X, wherein X can be any number from 1 to 135.

In some embodiments/aspects of the present invention, said polypeptide is one or more of the following: (a) said polypeptide belonging to the M20 Peptidase aminoacylase 1-like protein 2-like amidohydrolase subfamily (e.g., M20 Peptidase ACY1L2 amidohydrolase subfamily, e.g., having identifier cd05672 according to the Conserved Domain Database (e.g., https://www.ncbi.nlm.nih.gov/Structure/cdd/cddsrv.cgi?uid=cd05672), preferably said M20 Peptidase ACY1L2 amidohydrolase subfamily is characterized by having a metal (e.g. Zn) binding site, further preferably said metal binding site comprising an amino acid sequence motif selected from the group consisting of: CHEH[K,H] (SEQ ID NO: 20), CXHXXXXXXXXXXXXXXXXXXXXXXXXXXXXXXXXXXE (SEQ ID NO: 21) and CHEEH[K,H] (SEQ ID NO: 22); and/or (b) said polypeptide comprising: an amino acid C or an equivalent amino acid at a position corresponding to position 123 of SEQ ID NO: 1; an amino acid H or an equivalent amino acid at a position corresponding to position 125 of SEQ ID NO: 1; an amino acid E or an equivalent amino acid at a position corresponding to position 160 and/or 161 of SEQ ID NO: 1, preferably at a position corresponding to position 161 of SEQ ID NO: 1; an amino acid H or an equivalent amino acid at a position corresponding to position 185 of SEQ ID NO: 1; and an amino acid H or K, preferably H, or an equivalent amino acid at a position 383 of SEQ ID NO: 1; preferably using the numbering of SEQ ID NO: 1.

In some embodiments/aspects of the present invention, said polypeptide is not having aminopeptidase activity (e.g., EC 3.4.11.10) and/or (ii) said polypeptide is a non-peptidase and amidohydrolase.

In some embodiments/aspects of the present invention, said polypeptide is additionally capable of degrading and/or hydrolyzing a non-OTA (e.g., an OTA-unrelated) mycotoxin (e.g., trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- or bacteria-derived toxins (e.g. endotoxin, etc.).

In some embodiments/aspects, the present invention relates to a polynucleotide, nucleic acid construct or expression vector encoding and/or capable of expressing one or more polypeptides of the present invention.

In some embodiments/aspects, the present invention relates to a recombinant host cell (e.g., an isolated recombinant host cell), spore, transgenic plant, transgenic seed or transgenic pollen grain comprising one or more of the following: (i) one or more polypeptides of the present invention; (ii) one or more polynucleotides of the present invention; and/or (iii) one or more nucleic acid constructs and/or expression vectors of the present invention.

In some embodiments/aspects, the present invention relates to a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, comprising one or more of the following: one or more polypeptides, polynucleotides, nucleic acid constructs, expression vectors, recombinant host cells, spores, transgenic plants, transgenic seeds and/or transgenic pollen grains of the present invention.

In some embodiments/aspects, the present invention relates to a composition or kit comprising one or more of the following: polypeptides, polynucleotides, nucleic acid constructs, expression vectors, recombinant host cells, spores, transgenic plants, transgenic seeds and/or transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof of the present invention.

In some embodiments/aspects of the present invention, said composition or kit is one or more of the following: a cell-free, non-naturally occurring and/or fractionated composition or kit.

In some embodiments/aspects of the present invention, said composition or kit is further comprising one or more of the following: (i) one or more further polypeptides capable of detoxifying (e.g., modifying and/or hydrolyzing) and/or binding Ochratoxin A (OTA) and/or at least one OTA derivative (e.g., as defined herein, e.g., a compound or substance selected from the group consisting of: Furylacryloylphenylalanine (also called e.g. (2S)-2-[3-(furan-2-yl)prop-2-enoylamino]-3-phenylpropanoic acid), N-(3-(2-Furyl)acryloyl)phenylalanine (also called e.g. (2S)-2-[[(E)-3-(furan-2-yl)prop-2-enoyl]amino]-3-phenylpropanoic acid), L-Phenylalanine etc., preferably said at least one OTA derivative is ochratoxin B and/or ochratoxin C), further preferably said further one or more polypeptides comprise a carboxypeptidase activity (e.g., carboxypeptidase A and/or B activity, e.g., having EC 3.4.17.1 and/or EC 3.4.17.2 respectively) and/or thermolysin activity (e.g., having EC 3.4.24.27); (ii) one or more further polypeptides capable of detoxifying one or more further mycotoxins (e.g., trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- and/or bacteria-derived toxins (e.g. endotoxin, etc.), in particular said one or more further polypeptides capable of detoxifying one or more further mycotoxins and/or one or more plant- and/or bacteria-derived toxins, e.g., a fumonisin esterase (e.g. as disclosed in WO 2016/134387 A1) and/or a zearalenone lactonase (e.g. as disclosed in WO 2020/025580 A1) and/or an ergopeptine hydrolase (e.g. as disclosed in WO 2014/056006 A1); (iii) one or more organic absorbents (e.g., live, inactivated, lyophilized, dormant, and/or dead whole-yeast or yeast-derived product such as e.g. yeast cell wall, or yeast oligosaccharides such as e.g. mannan) and/or one or more inorganic absorbents (e.g., diatomaceous earth and/or clay mineral such as e.g. kaolins or kaolinites, smectites such as e.g. montmorillonites, illites or chlorites; in particular bentonite); (iv) one or more live, inactivated, lyophilized and/or dormant microorganisms capable of detoxifying one or more further mycotoxins (e.g., trichothecene mycotoxins such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- or bacteria-derived toxins (e.g. endotoxin, etc.), in particular said microorganism is selected from the group consisting of: Trichosporon and Apiotrichum genera (e.g. as disclosed in WO 03/053161 A1) and the Coriobacteriaceae family (e.g. as disclosed in EP 3 501 526 A1); (v) one or more plant products (e.g., seaweed, preferably seaweed meal; and/or algae, preferably algae meal; and/or thistle, preferably thistle seeds; and/or glycyrrhiza plant preparation, preferably glycyrrhiza meal and/or glycyrrhiza extract e.g. as disclosed in WO 2018/121881 A1); (vi) one or more flavoring compounds (e.g., plant extract e.g. from oregano, thyme, wintergreen, caraway, marjoram, mint, peppermint, anise, orange, lemon, fennel, star anise, clove, cinnamon and/or garlic; and/or essential oil such as e.g. D-limonene, γ-terpinene, p-cymene, 2-carene, linalool oxide, isomenthone, camphor, linalool, terpinen-4-ol, 2-isopropyl-1-methoxy-4-methylbenzene, L-menthol, ethylamine, α-terpineol, β-caryophyllene, D-carvone, methyl salicylate, α-caryophyllene, lavandulyl acetate, caryophyllene oxide, eugenol, thymol and/or carvacrol); (vii) one or more vitamins (e.g. vitamin A, D, E, K, C, B1, B2, B3, B4, B5, B6, B7, B8, B9 and/or B12; in particular vitamin E).

In particular, said composition or kit further comprises one or more of the following: bentonite, fumonisin esterase and/or a zearalenone lactonase, a Coriobacteriaceae microorganism (e.g., a microorganism selected from the family Coriobacteriaceae, e.g., https://lpsn.dsmz.de/family/coriobacteriaceae) capable of detoxifying one or more mycotoxins, diatomaceous earth, yeast (in particular, inactivated yeast), seaweed meal, thistle seeds, and one or more flavoring compound.

Further compositions (e.g., exemplary compositions 1-24 in Table II below) or kits (corresponding to exemplary compositions 1-24 in Table II below) of the present invention may comprise one or more further components in addition to at least one polypeptide according to the present invention. Such further exemplary compositions or kits are explicitly disclosed in Table II herein, which shows embodiments of the present invention.

TABLE II
Exemplary compositions or kits of the present invention comprising
one or more further components.

| Composition | | | | | | |
|---|---|---|---|---|---|---|
| Number | 1 | 2 | 3 | 4 | 5 | 6 |
| Any | yes | yes | yes | yes | yes | yes |
| polypeptide | | | | | | |
| according to | | | | | | |
| the present | | | | | | |
| invention | | | | | | |
| (e.g., SEQ ID | | | | | | |
| NOs: 1-4) | | | | | | |
| Any suitable | | | | e.g., | e.g., | |
| polypeptide | | | | fumonisin | fumonisin | |
| capable of | | | | esterase, | esterase, | |
| detoxifying | | | | e.g., having | e.g., | |
| one or more | | | | EC:3.1.1.87 | having | |
| further myco- | | | | enzymatic | EC:3.1.1.87 | |
| /plant- | | | | activity, | enzymatic | |
| /bacteria- | | | | e.g., | activity, | |
| toxins | | | | UniProtKB - | e.g., | |
| | | | | D2D3B6 | UniProtKB - | |
| | | | | and/or | D2D3B6 | |
| | | | | variant/s | and/or | |
| | | | | thereof | variant/s | |
| | | | | | thereof | |
| Any suitable | | e.g., | e.g., | e.g., | | |
| organic | | inactivated | dried yeast | yeast | | |
| absorbent | | yeast | | | | |
| Any suitable | e.g., | e.g., | e.g., | e.g., | e.g., | e.g., |
| inorganic | bentonite | zeolite | bentonite- | bentonite | bentonite | bentonite |
| absorbent | | | montmorillonite | | | |
| Any suitable | e.g., | | | e.g., | e.g., | |
| microorganism | Corio- | | | Corio- | Corio- | |
| capable of | bacteriaceae | | | bacteriaceae | bacteriaceae | |
| detoxifying | | | | | | |
| one or more | | | | | | |
| further | | | | | | |
| mycotoxins | | | | | | |
| Any suitable | | | e.g., | e.g., | | |
| plant product | | | algae | thistle seed | | |
| | | | powder | | | |
| Any suitable | | | | e.g., | | |
| vitamin | | | | Vitamin E | | |
| Any suitable | | | | yes | | |
| flavoring | | | | | | |
| compound | | | | | | |
| Composition | | | | | | |
| Number | 7 | 8 | 9 | 10 | 11 | 12 |
| Any | yes | yes | yes | yes | yes | yes |
| polypeptide | | | | | | |
| according to | | | | | | |
| the present | | | | | | |
| invention | | | | | | |
| (e.g., SEQ ID | | | | | | |
| NO: 1-4) | | | | | | |
| Any | | | e.g., | e.g., | e.g., | e.g., |
| polypeptide | | | fumonisin | fumonisin | fumonisin | fumonisin |
| capable of | | | esterase, | esterase, | esterase, | esterase, |
| detoxifying | | | e.g., having | e.g., having | e.g., | e.g., |
| one or more | | | EC:3.1.1.87 | EC:3.1.1.87 | having | having |
| further myco- | | | enzymatic | enzymatic | EC:3.1.1.87 | EC:3.1.1.87 |
| /plant- | | | activity, | activity, | enzymatic | enzymatic |
| /bacteria- | | | e.g., | e.g., | activity, | activity, |
| toxins | | | UniProtKB - | UniProtKB - | e.g., | e.g., |
| | | | D2D3B6 | D2D3B6 | UniProtKB - | UniProtKB - |
| | | | and/or | and/or | D2D3B6 | D2D3B6 |
| | | | variant/s | variant/s | and/or | and/or |
| | | | thereof | thereof | variant/s | variant/s |
| | | | | | thereof | thereof |
| Any suitable | | e.g., | | e.g., | | |
| organic | | inactivated | | Saccharomyces | | |
| absorbent | | yeast | | cerevisiae | | |
| Any suitable | e.g., | e.g., | e.g., | e.g., | e.g., | e.g., |
| inorganic | bentonite | bentonite | bentonite | bentonite | bentonite | bentonite |
| absorbent | | | | | | |
| Any suitable | e.g., | | e.g., | e.g., | e.g., | |
| microorganism | Eubacterium | | Corio- | Eubacterium | Corio- | |
| capable of | sp. | | bacteriaceae | sp. | bacteriaceae | |
| detoxifying | | | | | | |
| one or more | | | | | | |
| further | | | | | | |
| mycotoxin | | | | | | |
| Any suitable | e.g., | e.g., | | e.g., | | |
| plant product | algae | seaweed | | phycophytic | | |
| | extract | meal, | | substances | | |
| | | milk | | | | |
| | | thistle | | | | |
| Any suitable | | | | | | |
| vitamin | | | | | | |
| Any suitable | | | | | | |
| flavoring | | | | | | |
| compound | | | | | | |
| Composition | | | | | | |
| number | 13 | 14 | 15 | 16 | 17 | 18 |
| Any | yes | yes | yes | yes | yes | yes |
| polypeptide | | | | | | |
| according to | | | | | | |
| the present | | | | | | |
| invention | | | | | | |
| (e.g., SEQ ID | | | | | | |
| NOs: 1-4) | | | | | | |
| Any suitable | | | | e.g., | e.g., | |
| polypeptide | | | | fumonisin | fumonisin | |
| capable of | | | | esterase, | esterase, | |
| detoxifying | | | | e.g., having | e.g., | |
| one or more | | | | EC:3.1.1.87 | having | |
| further myco- | | | | enzymatic | EC:3.1.1.87 | |
| /plant- | | | | activity, e.g., | enzymatic | |
| /bacteria- | | | | UniProtKB - | activity, | |
| toxins | | | | D2D3B6 | e.g., | |
| | | | | and/or | UniProtKB - | |
| | | | | variant/s | D2D3B6 | |
| | | | | thereof, | and/or | |
| | | | | zearalenone | variant/s | |
| | | | | lactonase, | thereof, | |
| | | | | e.g., | zearalenone | |
| | | | | UniProtKB - | lactonase | |
| | | | | Q8NKB0 | e.g., | |
| | | | | and/or | UniProtKB - | |
| | | | | variant/s | Q8NKB0 | |
| | | | | thereof | and/or | |
| | | | | | variant/s | |
| | | | | | thereof | |
| Any suitable | | e.g., | e.g. | e.g., | | |
| organic | | inactivated | dried yeast | yeast | | |
| absorbent | | yeast | | | | |
| Any suitable | e.g. | e.g., | e.g., | e.g., | e.g., | e.g., |
| inorganic | bentonite | zeolite | bentonite- | bentonite | bentonite | bentonite |
| absorbent | | | montmorillonite | | | |
| Any suitable | e.g., | | | e.g., | e.g., | |
| microorganism | Corio- | | | Corio- | Corio- | |
| capable of | bacteriaceae | | | bacteriaceae | bacteriaceae | |
| detoxifying | | | | | | |
| one or more | | | | | | |
| further | | | | | | |
| mycotoxins | | | | | | |
| Any suitable | | | e.g., | | | |
| plant product | | | algae | | | |
| | | | powder | | | |
| Any suitable | | | | e.g., | | |
| vitamin | | | | Vitamin E | | |
| Any suitable | | | | yes | | |
| flavoring | | | | | | |
| compound | | | | | | |
| Composition | | | | | | |
| number | 19 | 20 | 21 | 22 | 23 | 24 |
| Any | yes | yes | yes | yes | yes | yes |
| polypeptide | | | | | | |
| according to | | | | | | |
| the present | | | | | | |
| invention | | | | | | |
| (e.g., SEQ ID | | | | | | |
| NOs: 1-4) | | | | | | |
| Any | | | fumonisin | fumonisin | fumonisin | fumonisin |
| polypeptide | | | esterase, | esterase, | esterase, | esterase, |
| capable of | | | e.g., having | e.g., having | e.g., | e.g., |
| detoxifying | | | EC:3.1.1.87 | EC:3.1.1.87 | having | having |
| one or more | | | enzymatic | enzymatic | EC:3.1.1.87 | EC:3.1.1.87 |
| further myco- | | | activity, e.g., | activity, e.g., | enzymatic | enzymatic |
| /plant- | | | UniProtKB - | UniProtKB - | activity, | activity, |
| /bacteria- | | | D2D3B6 | D2D3B6 | e.g., | e.g., |
| toxins | | | and/or | and/or | UniProtKB - | UniProtKB - |
| | | | variant/s | variant/s | D2D3B6 | D2D3B6 |
| | | | thereof, | thereof, | and/or | and/or |
| | | | zearalenone | zearalenone | variant/s | variant/s |
| | | | lactonase, | lactonase, | thereof, | thereof, |
| | | | e.g., | e.g., | zearalenone | zearalenone |
| | | | UniProtKB - | UniProtKB - | lactonase, | lactonase, |
| | | | Q8NKB0 | Q8NKB0 | e.g., | e.g., |
| | | | and/or | and/or | UniProtKB - | UniProtKB - |
| | | | variant/s | variant/s | Q8NKB0 | Q8NKB0 |
| | | | thereof | thereof | and/or | and/or |
| | | | | | variant/s | variant/s |
| | | | | | thereof | thereof |
| Any suitable | | e.g., | | e.g., | | |
| organic | | inactivated | | Saccharomyces | | |
| absorbent | | yeast | | cerevisiae | | |
| Any suitable | e.g., | e.g., | e.g., | e.g., | e.g., | e.g., |
| inorganic | bentonite | bentonite | bentonite | bentonite | bentonite | bentonite |
| absorbent | | | | | | |
| Any suitable | e.g., | | e.g., | e.g., | e.g., | |
| microorganism | Eubacterium | | Corio- | Eubacterium | Corio- | |
| capable of | sp. | | bacteriaceae | sp. | bacteriaceae | |
| detoxifying | | | | | | |
| one or more | | | | | | |
| further | | | | | | |
| mycotoxins | | | | | | |
| Any suitable | e.g., | e.g., | | e.g., | | |
| plant product | algae | seaweed | | phycophytic | | |
| | extract | meal, | | substances | | |
| | | milk | | | | |
| | | thistle | | | | |
| Any suitable | | | | | | |
| vitamin | | | | | | |
| Any suitable | | | | | | |
| flavoring | | | | | | |
| compound | | | | | | |

In some embodiments/aspects of the present invention, said composition or kit is a pharmaceutical, veterinary, diagnostic, detoxifying, monitoring and/or screening composition or kit.

In some embodiments/aspects, the present invention relates to method for producing a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, said method comprising: (a) providing: (i) one or more polypeptides of the present invention; (ii) one or more polynucleotides of the present invention; (iii) one or more nucleic acid constructs and/or expression vectors of the present invention and/or (iv) one or more recombinant host cells, spores, transgenic plants, transgenic seeds and/or transgenic pollen grains of the present invention; preferably said one or more polypeptides are one or more recombinant and/or isolated polypeptides; (b) applying (a) to a nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof.

In some embodiments/aspects of the present invention, said method further comprising: incubating said nutritive source or material with (a) under conditions suitable for detoxifying ochratoxin A (OTA) and/or at least one ochratoxin A derivative and/or altering toxicity of OTA and/or OTA derivative/s; preferably said method further comprising heat-treating and/or fractionating and/or drying the product of said incubation.

In some embodiments/aspects, the present invention relates to a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof produced by the method of the present invention.

In some embodiments/aspects, the present invention relates to a method for detoxifying ochratoxin A (OTA) and/or altering toxicity of OTA and/or at least one OTA derivative, said method comprising: (a) providing: (i) one or more polypeptides according to any one of the preceding items; (ii) one or more polynucleotides according to any one of the preceding items; (iii) one or more nucleic acid constructs and/or expression vectors according to any one of the preceding items and/or (iv) one or more recombinant host cells, spores, transgenic plants, transgenic seeds and/or transgenic pollen grains according to any one of the preceding items; preferably said one or more polypeptides are one or more recombinant and/or isolated polypeptides; (b) applying (a) to OTA and/or to at least one OTA derivative.

In some embodiments/aspects the method of the present invention is an in vitro or ex vivo method.

In some embodiments/aspects of the present invention, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit of the present invention can be used for/in therapy, prophylaxis and/or as a medicament (e.g., for veterinary use).

In some embodiments/aspects of the present invention, polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit of the present invention can be used for/in one or more of the following methods: method for treatment, amelioration, prophylaxis and/or diagnostics of mycotoxicosis, preferably an OTA mycotoxicosis; method for monitoring development of mycotoxicosis and/or assessing the efficacy of a mycotoxicosis prophylaxis and/or therapy, preferably an OTA mycotoxicosis prophylaxis and/or therapy; method for detoxifying ochratoxin A (OTA) and/or altering toxicity of OTA and/or at least one OTA derivative; method of producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit; any method of the present invention; any combination of methods as described above; any method as described above, wherein said method is an in vitro, ex vivo or in vivo method.

In some embodiments/aspects, the present invention relates to a use of one or more of polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding items for/in one or more of the following: treatment, amelioration, prophylaxis and/or diagnostics of mycotoxicosis, preferably OTA mycotoxicosis; monitoring development of mycotoxicosis and/or assessing the efficacy of a mycotoxicosis prophylaxis and/or therapy, preferably OTA mycotoxicosis prophylaxis and/or therapy; detoxifying ochratoxin A (OTA) and/or altering toxicity of OTA and/or at least one OTA derivative; producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit; in any method as described above; any combination of methods as described above, any use as described above, wherein said use is an in vitro, ex vivo or in vivo use.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is also characterized by the following items:
1. A polypeptide capable of detoxifying (e.g., modifying or hydrolyzing) Ochratoxin A

(OTA) and/or at least one OTA derivative (e.g., as listed above, e.g., a compound or substance selected from the group consisting of: Furylacryloylphenylalanine (also called e.g. (2S)-2-[3-(furan-2-yl)prop-2-enoylamino]-3-phenylpropanoic acid), N-(3-(2-Furyl)acryloyl)phenylalanine (also called e.g. (2S)-2-[[(E)-3-(furan-2-yl)prop-2-enoyl]amino]-3-phenylpropanoic acid), L-Phenylalanine etc., preferably said at least one OTA derivative is ochratoxin B and/or ochratoxin C), wherein said polypeptide is one or more of the following:
a) a polypeptide having at least 60% sequence identity (e.g. at least 64%, 65%, 66%, 67%, 68%, 69%, 70%, 71%, 72%, 73%, 74%, 80%, 81%, 82%, 83%, 84%, 85%, 86%, 87%, 88%, 89%, 90%, 91%, 92%, 93%, 94%, 95%, 96%, 97%, 98%, 99% or 100%) to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-4; further preferably said polypeptide having SEQ ID NO: 1;
b) a variant of the polypeptide (a), wherein said variant comprising a substitution, deletion, and/or insertion at one or more positions (e.g., said variant having one or more conservative or equivalent substitutions, e.g., as shown in Table I above);
c) a fragment of the polypeptide of (a) or (b), wherein said fragment is capable of detoxifying OTA and/or at least one OTA derivative; d) a polypeptide comprising one or more of the following amino acid sequences (e.g., conservative motifs):

i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SE ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGSPAEEGGGGKX
ILX NEGXYDXXDACXMAHPXGG, wherein X is any amino
acid;
xvi)
(i), (ii), (iii) and (iv) (e.g., SEQ ID NOs: 5, 6,
7 and 23);
xvii)
(SEQ ID NO: 8)
AHAGAAPW.

2. The polypeptide according to any one of the preceding items, wherein said polypeptide is selected from the group consisting of: SEQ ID NOs: 1-4; preferably said polypeptide is selected from the group consisting of: SEQ ID NOs: 1-3; further preferably said polypeptide having at least 69% sequence identity to the amino acid sequence of SEQ ID NO: 1 (e.g., SEQ ID NO: 2 is 69.1% identical to SEQ ID NO: 1; SEQ ID NO: 3 is 69.3% identical to SEQ ID NO: 1); most preferably said polypeptide having SEQ ID NO: 1.
3. The polypeptide according to any one of the preceding items, wherein said polypeptide is capable/having of: (i) degrading ochratoxin A with a specific activity of at least 0.01 U/g (e.g. as shown in Example 2 herein, e.g., at least 0.05, 0.1, 0.5, 0.75, 1.0, 1.2, 2, 5, 7 or 10 U/g); (ii) thermostability (e.g., a temperature range in which 50% of the initial activity was lost, e.g., as shown in Example 3 herein, e.g., in Table 2, e.g., above 55° C.); and/or (iii) suitable for OTA detoxification of food and/or feed (e.g., as shown in Example 4 herein).
4. The polypeptide according to any one of the preceding items, wherein said polypeptide comprising one or more of the following amino acid sequences (e.g., amino acid motifs, e.g., conservative motifs):

i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGSPAEEGGGGKXIL
XNEGXYDXXDACXMAHPXGG,
wherein X is any amino acid;
xvi)
(SEQ ID NO: 8)
AHAGAAPW;

xvii) a polypeptide having at least 70% sequence identity (e.g. at least 70%, 74%, 80%, 90%, 91%, 92%, 93%, 94%, 95%, 96%, 97%, 98%, 99%, 100%) to the amino acid sequence selected from the group consisting (i)-(xvi);
xviii) a polypeptide having at least 70% identity to the amino acid sequence of (xv); and/or having at least 80% identity to any one of (viii)-(xiv); and/or having at least 90% to (vi) and/or (vii);
xix) a variant of the polypeptide having amino acid sequence (i), (ii), (iii), (iv), (v), (vi), (vii), (viii), (ix), (x), (xi), (xii), (xiii) or (xiv), wherein said variant comprises a substitution, deletion, and/or insertion at one or more positions;)
xx) a polypeptide comprising amino acid sequences: (i), (ii), (iii) and (iv).
5. The polypeptide according to any one of the preceding items, wherein said detoxifying Ochratoxin A (OTA) comprising or consisting of hydrolyzing an amide bond of OTA.
6. The polypeptide according to any one of the preceding items, wherein said polypeptide having amidohydrolase activity (e.g., EC 3.5.1.-, e.g., EC 3.5.1.14 (aminoacylase 1)).
7. The polypeptide according to any one of the preceding items, wherein said polypeptide is one or more of the following:
   a) said polypeptide belonging to the M20 Peptidase aminoacylase 1-like protein 2-like amidohydrolase subfamily (e.g., M20 Peptidase ACY1L2 amidohydrolase subfamily, e.g., having identifier cd05672 according to the Conserved Domain Database), preferably said M20 Peptidase ACY1L2 amidohydrolase subfamily is characterized by having a metal (e.g. Zn) binding site, further preferably said metal binding site comprising an amino acid sequence motif selected from the group consisting of: CHEH[K,H] (SEQ ID NO: 20), CXHXXXXXXXXXXXXXXXXXXXXXXXXXXXXXXXXXXE (SEQ ID NO: 21) and CHEEH[K,H] (SEQ ID NO: 22); and/or
   b) said polypeptide comprising: an amino acid C or an equivalent amino acid at a position corresponding to position 123 of SEQ ID NO: 1; an amino acid H or an equivalent amino acid at a position corresponding to position 125 of SEQ ID NO: 1; an amino acid E or an equivalent amino acid at a position corresponding to position 160 and/or 161 of SEQ ID NO: 1, preferably at a position corresponding to position 161 of SEQ ID NO: 1; an amino acid H or an equivalent amino acid at a position corresponding to position 185 of SEQ ID NO: 1; and an amino acid H or K, preferably H, or an equivalent amino acid at a position 383 of SEQ ID NO: 1; preferably using the numbering of SEQ ID NO: 1.
8. The polypeptide according to any one of the preceding items, wherein: (i) said polypeptide is not having aminopeptidase activity (e.g., EC 3.4.11.10) and/or (ii) wherein said polypeptide is a non-peptidase amidohydrolase.
9. The polypeptide according to any one of the preceding items, wherein said polypeptide is capable of degrading and/or hydrolyzing non-OTA (e.g., OTA-unrelated) mycotoxin, e.g., trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- or bacteria-derived toxins (e.g. endotoxin, etc.).
10. A polynucleotide, nucleic acid construct or expression vector encoding and/or capable of expressing one or more polypeptides according to any one of the preceding items.
11. A recombinant host cell (e.g., an isolated recombinant host cell), spore, transgenic plant, transgenic seed or transgenic pollen grain comprising one or more of the following: (i) one or more polypeptides according to any one of the preceding items; (ii) one or more polynucleotides according to any one of the preceding items; and/or (iii) one or more nucleic acid constructs and/or expression vectors according to any one of the preceding items.
12. A foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, comprising one or more of the following: one or more polypeptides, polynucleotides, nucleic acid constructs, expression vectors, recombinant host cells, spores, transgenic plants, transgenic seeds and/or transgenic pollen grains according to any one of the preceding items.
13. A composition or kit comprising one or more of the following: polypeptides, polynucleotides, nucleic acid constructs, expression vectors, recombinant host cells, spores, transgenic plants, transgenic seeds and/or transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement, prebiotic, intermediate prebiotic and/or mixture/s thereof according to any one of the preceding items.
14. The composition or kit according to any one of the preceding items, wherein said composition or kit is one or more of the following: a cell-free, non-naturally occurring and/or fractionated composition or kit.
15. The composition or kit according to any one of the preceding items, wherein said composition or kit is further comprising one or more of the following (e.g., Table II): (i) one or more further polypeptides capable of detoxifying (e.g., modifying and/or hydrolyzing) and/or binding Ochratoxin A (OTA) and/or at least one OTA derivative (e.g., as defined herein, e.g., a compound or substance selected from the group consisting of: Furylacryloylphenylalanine (also called e.g. (2S)-2-[3-(furan-2-yl)prop-2-enoylamino]-3-phenylpropanoic acid), N-(3-(2-Furyl)acryloyl)phenylalanine (also called e.g. (2S)-2-[[(E)-3-(furan-2-yl)prop-2-enoyl]amino]-3-phenylpropanoic acid), L-Phenylalanine etc., preferably said at least one OTA derivative is ochratoxin B and/or ochratoxin C), preferably said further one or more polypeptides comprise a carboxypeptidase activity (e.g., carboxypeptidase A and/or B activity, e.g., having EC 3.4.17.1 and/or EC 3.4.17.2 respectively) and/or thermolysin activity (e.g., having EC 3.4.24.27); (ii) one or more further polypeptides capable of detoxifying one or more further mycotoxins and/or one or more plant- and/or bacteria-derived toxins (e.g., trichothecene mycotoxin/s such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- and/or bacteria-derived toxins (e.g. endotoxin, etc.), in particular said one or more further polypeptides capable of detoxifying one or more further mycotoxins and/or one or more plant- and/or bacteria-derived toxins, e.g., a fumonisin esterase (e.g. as disclosed in WO 2016/134387 A1) and/or a zearalenone lactonase (e.g. as disclosed in WO 2020/025580 A1) and/or an ergopeptine hydrolase (e.g. as disclosed in WO 2014/056006 A1); (iii) one or more organic absorbents (e.g., live/inactivated/lyophilized/dormant/dead whole-yeast or yeast-derived product such as e.g. yeast cell wall, or yeast oligosaccharides such as e.g. mannan) and/or inorganic absorbant (e.g., diatomaceous earth and/or clay mineral such as e.g. kaolins or kaolinites, smectites such as e.g. montmorillonites, illites or chlorites; in particular bentonite); (iv) one or more live, inactivated, lyophilized and/or dormant microorganisms capable of detoxifying one or more further mycotoxin (e.g., trichothecene mycotoxins such as e.g. deoxynivalenol, nivalenol, neosolaniol, trichotecin, crotocin, roridin A, satratoxin H, diacetoxyscirpenol, HT-2 toxin or T-2 toxin; aflatoxins such as e.g. aflatoxin B1, B2, G1 or G2; fumonisins such as e.g. fumonisin B1, B2, B3 or B4; polypeptide mycotoxins such as e.g. beauvericin or enniatins; zearalenone; citrinin; patulin; ergot alkaloids such as e.g. ergotamine) and/or one or more plant- or bacterial-derived toxin (e.g. endotoxin), in particular said microorganism is a member of the Trichosporon or Apiotrichum genus (e.g. as disclosed in WO 03/053161 A1) or of the Coriobacteriaceae family (e.g. as disclosed in EP 3 501 526 A1); (v) one or more plant product (e.g., seaweed, preferably seaweed meal; and/or algae, preferably algae meal; and/or thistle, preferably thistle seeds; and/or glycyrrhiza plant preparation, preferably glycyrrhiza meal and/or glycyrrhiza extract e.g. as disclosed in WO 2018/121881 A1); (vi) one or more flavoring compound (e.g., plant extract e.g. from oregano, thyme, wintergreen, caraway, marjoram, mint, peppermint, anise, orange, lemon, fennel, star anise, clove, cinnamon and/or garlic; and/or essential oil such as e.g. D-limonene, γ-terpinene, p-cymene, 2-carene, linalool oxide, isomenthone, camphor, linalool, terpinen-4-ol, 2-isopropyl-1-methoxy-4-methylbenzene, L-menthol, ethylamine, α-terpineol, β-caryophyllene, D-carvone, methyl salicylate, α-caryophyllene, lavandulyl acetate, caryophyllene oxide, eugenol, thymol and/or carvacrol); (vii) one or more vitamin (e.g. vitamin A, D, E, K, C, B1, B2, B3, B4, B5, B6, B7, B8, B9, B12; in particular vitamin E).
16. The composition or kit according to any one of the preceding items, wherein said composition or kit is a pharmaceutical, veterinary, diagnostic, detoxifying, monitoring and/or screening composition or kit.
17. A method for producing a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, said method comprising:
   a) providing: (i) one or more polypeptides according to any one of the preceding items; (ii) one or more polynucleotides according to any one of the preceding items; (iii) one or more nucleic acid constructs and/or expression vectors according to any one of the preceding items and/or (iv) one or more recombinant host cells, spores, transgenic plants, transgenic seeds and/or transgenic pollen grains according to any one of the preceding items; preferably said one or more polypeptides are one or more recombinant and/or isolated polypeptides;
   b) applying (a) to a nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof.
18. The method according to any one of the preceding items, wherein said method further comprising: incubating said nutritive source or material with (a) under conditions suitable for detoxifying ochratoxin A (OTA) and/or at least one ochratoxin A derivative and/or altering toxicity of OTA and/or OTA derivative/s; preferably said method further comprising heat-treating and/or fractionating and/or drying the product of said incubation.
19. A foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof produced by the method according to any one of the preceding items.
20. A method for detoxifying ochratoxin A (OTA) and/or altering toxicity of OTA and/or at least one OTA derivative, said method comprising:
   a) providing: (i) one or more polypeptides according to any one of the preceding items; (ii) one or more polynucleotides according to any one of the preceding items; (iii) one or more nucleic acid constructs and/or expression vectors according to any one of the preceding items and/or (iv) one or more recombinant host cells, spores, transgenic plants, transgenic seeds and/or transgenic pollen grains according to any one of the preceding items; preferably said one or more polypeptides are one or more recombinant and/or isolated polypeptides;
   b) applying (a) to OTA and/or to at least one OTA derivative.
21. The polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding items, for use in therapy, prophylaxis and/or as a medicament (e.g., for veterinary use).
22. The polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding items, for use in one or more of the following methods:
   (i) method for treatment, amelioration, prophylaxis and/or diagnostics of mycotoxicosis, preferably an OTA mycotoxicosis;
   (ii) method for monitoring development of mycotoxicosis and/or assessing the efficacy of a mycotoxicosis prophylaxis and/or therapy, preferably an OTA mycotoxicosis prophylaxis and/or therapy;
   (iii) method for detoxifying ochratoxin A (OTA) and/or altering toxicity of OTA and/or at least one OTA derivative;
   (iv) method of producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit;
   (v) method according to any one of the preceding items;
   (vi) any combination of methods according to (i)-(v);
   (vii) method of any of (i)-(vi), wherein said method is an in vitro, ex vivo or in vivo method.
23. Use of one or more of polypeptide, polynucleotide, nucleic acid construct, expression vector, recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding items for/in one or more of the following:
   (i) treatment, amelioration, prophylaxis and/or diagnostics of mycotoxicosis, preferably OTA mycotoxicosis;
   (ii) monitoring development of mycotoxicosis and/or assessing the efficacy of a mycotoxicosis prophylaxis and/or therapy, preferably OTA mycotoxicosis prophylaxis and/or therapy;
   (iii) detoxifying ochratoxin A (OTA) and/or altering toxicity of OTA and/or at least one OTA derivative;
   (iv) producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit;
   (v) in method according to any one of the preceding items;
   (vi) any combination of according to (i)-(v);
   (vii) use of any of (i)-(vi), wherein said use is an in vitro, ex vivo or in vivo use.
24. A method for producing a foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, said method comprising:
   a) providing: (i) one or more polypeptides according to any one of the preceding items; (ii) one or more polynucleotides according to any one of the preceding items; (iii) one or more nucleic acid constructs and/or expression vectors according to any one of the preceding items and/or (iv) one or more recombinant host cells, spores, transgenic plants, transgenic seeds and/or transgenic pollen grains according to any one of the preceding items; preferably said one or more polypeptides are one or more recombinant and/or isolated polypeptides;
   b) applying (a) to a nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof.
25. The method according to any one of the preceding items, said method comprising:
   a) providing:
      (a′) one or more polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
         (aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
         (aaa′) a polypeptide comprising one or more of the following amino acid sequences (e.g., conservative motifs):

i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;

(b′) one or more polynucleotides encoding and/or capable of expressing one or more polypeptides according to (a′);
(c′) one or more nucleic acid constructs and/or expression vectors encoding and/or capable of expressing one or more polypeptides according to (a′); and/or
(d′) one or more recombinant host cells, spores, transgenic plants, transgenic seeds and/or transgenic pollen grains comprising one or more of the following: (i′) one or more polypeptides according to (a′); (ii′) one or more polynucleotides according to (b′); and/or (iii′) one or more nucleic acid constructs and/or expression vectors according to (c′); preferably said one or more polypeptides are one or more recombinant and/or isolated polypeptides;
b) applying (a) to a nutritive source or material suitable for production of foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof.
26. A method for detoxifying ochratoxin A (OTA) and/or altering toxicity of OTA and/or at least one OTA derivative, said method comprising:
   a) providing: (i) one or more polypeptides according to any one of the preceding items; (ii) one or more polynucleotides according to any one of the preceding items; (iii) one or more nucleic acid constructs and/or expression vectors according to any one of the preceding items and/or (iv) one or more recombinant host cells, spores, transgenic plants, transgenic seeds and/or transgenic pollen grains according to any one of the preceding items; preferably said one or more polypeptides are one or more recombinant and/or isolated polypeptides;
   b) applying (a) to OTA and/or to at least one OTA derivative.
27. The method according to any one of the preceding items, said method comprising:
   a) providing:
      (a′) one or more polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
         (aa') a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
         (aaa′) a polypeptide comprising one or more of the following amino acid sequences (e.g., conservative motifs):

i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;

(b′) one or more polynucleotides encoding and/or capable of expressing one or more polypeptides according to (a′);
(c′) one or more nucleic acid constructs and/or expression vectors encoding and/or capable of expressing one or more polypeptides according to (a′); and/or
(d′) one or more recombinant host cells, spores, transgenic plants, transgenic seeds and/or transgenic pollen grains comprising one or more of the following: (i′) one or more polypeptides according to (a′); (ii′) one or more polynucleotides according to (b′); and/or (iii′) one or more nucleic acid constructs and/or expression vectors according to (c′); preferably said one or more polypeptides are one or more recombinant and/or isolated polypeptides;
b) applying (a) to OTA and/or to said at least one OTA derivative.
28. The method according to any one of the preceding items, wherein said polypeptide, polynucleotide, nucleic acid construct, expression vector and/or recombinant host cell, spore, transgenic plant, transgenic seed and/or transgenic pollen grain is according to any one of the preceding items.

The invention is further illustrated by the following examples, however, without being limited to the example or by any specific embodiment of the examples.

### EXAMPLES OF THE INVENTION

### Example 1: Cloning of Genes and Recombinant Polypeptide Production

Genes encoding either of the polypeptide sequences SEQ ID NOs: 1-4 were synthesized by the commercial provider TWIST Bioscience (https://www.twistbioscience.com/) with codons optimized for expression in Escherichia coli. The genes were initially designed to further encode a hexahistidine tag C-terminally fused to either of the polypeptide sequences to facilitate later purification. Subsequent polypeptide productions and enzyme characterizations were also performed with untagged enzymes yielding essentially identical results.

The genes were cloned into expression vectors using common tools for expression in E. coli as known in the art. In particular, the T7 promoter system with a lac operator (Dubendorf and Studier 1991, J. Mol. Biol. 219, 45-59) was used to regulate gene expression. An E. coli BL21(DE3) strain was transformed with said expression vectors. A kanamycin resistance marker was used to allow selection of transformants on agar plates containing 50 μg/mL kanamycin.

For recombinant protein production, single transformant colonies were incubated over night in terrific broth (TB) medium (containing 12 g/L tryptone, 24 g/L yeast extract, 5 g/L glycerol, 50 mg/L kanamycin) at 37° C. under shaking to produce a preculture. After incubation, 1 mL of this preculture was added to 50 mL of fresh TB medium and incubation was continued as before until an OD600 of 0.5-0.8 was reached. Subsequently, isopropyl-beta-D-thiogalactoside (IPTG) was added to a final concentration of 400 μM to initiate gene expression. Gene expression was performed by further incubation for 18-20 h at 26-28° C. while shaking. Thereafter, cells were harvested by centrifugation. The supernatant was discarded and the cell pellet was resuspended in 50 mM Tris-HCl buffer, pH 7.5, prior to cell disruption on ice using an ultrasonication system (QSonica). Crude lysate was cleared by centrifugation (18 min, 21, 130×g, 4° C.). The presence of recombinant target enzyme in the soluble fraction of the lysate was verified by SDS-PAGE (Laemmli 1970, Nature 227, 680-685) using Bio-Rad 12% mini-PROTEAN TGX stain-free precast gels. Alternative strategies suitable for achieving recombinant protein production either in further E. coli strains, in other bacterial production hosts (e.g. Bacillus subtilis, Corynebacterium glutamicum etc.) as well as in other eukaryotic hosts (e.g. CHO cells, yeasts e.g. Saccharomyces cerevisiae, Pichia pastoris etc., other fungal hosts e.g. Aspergillus spp., Trichoderma spp. etc.) are described in the state-of-the-art and well-known to a skilled person.

For further purification and isolation of hexahistidine-tagged recombinant enzyme by affinity chromatography, His GraviTrap TALON columns (GE Healthcare) were used. To this end, cleared lysate was loaded onto the column, followed by washing with wash buffer containing 10 mM imidazole, 300 mM NaCl, to remove non-target polypeptides. Recombinant target protein bound to the column was eluted with elution buffer containing 150 mM imidazole, 300 mM NaCl. The imidazole contained in the eluate was removed by washing with 50 mM Tris-HCl buffer, pH 7.5, and concomitant enzyme concentration using VivaSpin 6 centrifugation columns (Sartorius). Purified and concentrated enzyme was again analyzed by SDS-PAGE using the software GelAnalyzer (e.g., 2010a version) to verify the purity of the preparation and to estimate protein concentration by comparison with bovine serum albumin (BSA) standards of known concentrations.

### Example 2: Determination of Specific Activity

To determine the specific activity of recombinant target polypeptides produced e.g. as described in Example 1 above in units per liter (U/L), ochratoxin A (OTA) hydrolyzing activity assays were performed. One unit is defined as the amount of enzyme necessary for hydrolysis of 1 μmol or OTA in 1 min when using a starting OTA concentration of 0.495 μM.

OTA hydrolyzing activity assays were set up in a final volume of the reaction mixture of 200 μL, containing the recombinant enzyme preparation (preferably diluted to yield a volumetric activity of 1-10 mU/L), and 200 ng/mL OTA in 100 mM sodium phosphate buffer.

Specific activities were determined at pH 6.0 and pH 7.5. As soon as all components of the reaction mixture were mixed, OTA hydrolysis was followed at 37° C. on a fluorescence spectrophotometer (BioTek Synergy H1 MFD Multimode microplate reader) by following the reduction in fluorescence at ex/em 390 nm/450 nm.

For calculation of the specific activity, the initial linear part of the recorded fluorescence reduction curve was used. In addition to the fluorescence-based method described above, OTA hydrolysis was also determined by using the HPLC-MS method as described by Dellafiora et al. 2020, Toxins 12, 258, essentially confirming the fluorescence-based results. The specific activities for the polypeptides having amino acid sequences of SEQ ID NOs: 1-4 at different pH are shown in Table 1.

TABLE 1
Specific OTA hydrolyzing activities in U/g at different pH.

| SEQ ID No: | pH 6 | pH 7.5 |
|---|---|---|
| 1 | 0.11 | 1.20 |
| 2 | 3.53 | 11.01 |
| 3 | 0.63 | 1.44 |
| 4 | 1.57 | 1.43 |

### Example 3: Determination of Thermostability

To determine the kinetic stability of the produced enzymes towards temperature, thermostability assays were performed. To this end, purified protein samples were diluted to a protein concentration of 0.1 mg/mL in 20 mM Tris-HCl buffer pH 7.5 and aliquots were incubated in a thermocycler for 10 min at either 45, 55, 65, 75 or 85 ° C. After this incubation, the aliquots were stored at 4° C. until further analysis. A reference aliquot was stored at 4° C. throughout the incubation. Subsequently, all aliquots were assayed for residual OTA hydrolyzing activity using the assay described in Example 2. Residual enzyme activities were calculated relative to the reference aliquot. The temperature range in which 50% of the initial activity was lost after the incubation, in relation to the reference aliquot was determined. The results are shown in Table 2.

TABLE 2
Temperature range in which 50% of the initial activity was lost.

| SEQ ID No: | Temperature |
|---|---|
| 1 | 45-55° C. |
| 2 | 65-75° C. |
| 3 | 55-65° C. |
| 4 | 45-55° C. |

### Example 4: Feeding Trial

To verify the applicability of the produced enzymes as food or feed additive, piglet feeding trials were conducted. To this end, sufficient amounts of the polypeptide of SEQ ID NO: 1 were produced by cultivation of E. coli transformants in bioreactors. The cell broth was homogenized and centrifuged. The supernatant was concentrated, dried by lyophilization and formulated as a powder by grinding. The enzymatic activity of the thus obtained lyophilized powder was determined, and the powder was mixed with feed for the feeding trial as described in the following.

Five piglet groups were studied based on the following five experimental diet setups: 1) Base feed; 2) base feed+50 ppb OTA; 3) base feed+50 ppb OTA+169 mU of the enzyme of SEQ ID NO: 1 per kg of feed; 4) base feed+500 ppb OTA; 5) base feed+500 ppb OTA+169 mU of the enzyme of SEQ ID NO: 1 per kg of feed. The base feed itself did not contain any detectable amount of OTA.

Each trial group consisted of four piglets weighing approximately 7-8 kg, who were coming out from weaning and adapted to solid diet before being fed the experimental diet as described above. The duration of the feeding trial (i.e. actual days of feeding the experimental diet) was fourteen days. Collectively each trial group consumed around 15 kg of feed. No piglets fell sick to any unexpected disease throughout the feeding trial. Urine and blood samples were collected on day 0, just before starting the experimental diet, and on day 14.

Extraction of OTA was performed from plasma and urine samples for subsequent analysis by HPLC-MS. 200 μL of sample was thoroughly mixed with 800 μL of extraction solution (containing 99% of ethyl acetate and 1% of phosphoric acid, 85%) followed by centrifugation at 19000×g for 5 min for phase separation. 50 μL of upper phase was diluted with 50 μL of acetonitrile, followed by HPLC-MS analysis. The H PLC-MS method as described by Dellafiora et al. 2020, Toxins 12, 258 was used to determine the changes in OTA concentration in the samples taken during the trial upon consumption of the experimental diets. The results are shown in Table 3 using the diet setup numbering as indicated above. Notably, a reduction of the OTA concentration in both, plasma and urine, was observed only when recombinant enzyme was part of the diet setup, demonstrating the applicability of the enzymes of the present invention in food/feed for OTA detoxification.

TABLE 3
OTA concentrations in plasma and blood samples on
day 0 and day 14.

| Diet | Plasma samples | | | Urine samples | | |
|---|---|---|---|---|---|---|
| setup | | | % OTA | | | % OTA |
| # | Day 0 | Day 14 | reduction | Day 0 | Day 14 | reduction |
| 1 | 0 | 0 | n/a | 0 | 0.00 | n/a |
| 2 | 0 | 41 ± 4 | n/a | 0 | 4.25 | n/a |
| 3 | 0 | 37 ± 3 | 9 | 0 | 0.00 | 100.0 |
| 4 | 0 | 414 ± 72 | n/a | 0 | 48.79 | n/a |
| 5 | 0 | 350 ± 25 | 15 | 0 | 25.18 | 48.4 |

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge. The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes. Other embodiments are within the following claims. In addition, where features or aspects of the invention are described in terms of Markush groups, those skilled in the art will recognize that the invention is also thereby described in terms of any individual member or subgroup of members of the Markush group.

1. A method for producing a foodstuff, intermediate foodstuff; fodder, intermediate fodder;
feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, said method comprising:
a) providing:
(a′) one or more polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
(b′) one or more polynucleotides encoding and/or capable of expressing one or more polypeptides according to (a′);
(c′) one or more nucleic acid constructs and/or expression vectors encoding and/or capable of expressing one or more polypeptides according to (a′); and/or
(d′) one or more recombinant host cells, spores, transgenic plants, transgenic seeds and/or transgenic pollen grains comprising one or more of the following: (i′) one or more polypeptides according to (a′); (ii′) one or more polynucleotides according to (b′); and/or (iii′) one or more nucleic acid constructs and/or expression vectors according to (c′); preferably said one or more polypeptides are one or more recombinant and/or isolated polypeptides;
b) applying (a) to a nutritive source or material suitable for production of foodstuff, intermediate foodstuff; fodder, intermediate fodder; feed, intermediate feed;
additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement;
prebiotic, intermediate prebiotic and/or mixture/s thereof.

2. A method for detoxifying ochratoxin A (OTA) and/or altering toxicity of OTA and/or at least one OTA derivative, wherein said OTA derivative is preferably selected from the group consisting of Ochratoxin B and Ochratoxin C, said method comprising:
a) providing:
(a′) one or more polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG;
wherein X is any amino acid;
xvi)
(i), (ii), (iii) and (iv);
and/or
xvii)
(SEQ ID NO: 8)
AHAGAAPW;
(b′) one or more polynucleotides encoding and/or capable of expressing one or more polypeptides according to (a′);
(c′) one or more nucleic acid constructs and/or expression vectors encoding and/or capable of expressing one or more polypeptides according to (a′); and/or
(d′) one or more recombinant host cells, spores, transgenic plants, transgenic seeds and/or transgenic pollen grains comprising one or more of the following: (i′) one or more polypeptides according to (a′); (ii′) one or more polynucleotides according to (b′); and/or (iii′) one or more nucleic acid constructs and/or expression vectors according to (c′); preferably said one or more polypeptides are one or more recombinant and/or isolated polypeptides;
b) applying (a) to OTA and/or to said at least one OTA derivative.

3. A polypeptide capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following:
a) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2 and SEQ ID NO: 3; preferably said polypeptide having an amino acid sequence selected from the group consisting of: SEQ ID NOs: 1-3; further preferably said polypeptide having SEQ ID NO: 1;
b) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGSPAEEGGGGKXIL
XNEGXYDXXDACXMAHPXGG,
wherein X is any amino acid;
xvi)
(i), (ii), (iii) and (iv);
and/or
xvii)
(SEQ ID NO: 8)
AHAGAAPW

4. The polypeptide according to any one of the preceding claims, wherein said polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGSPAEEGGGGKXIL
XNEGXYDXXDACXMAHPXGG,
wherein X is any amino acid;
xvi)
(SEQ ID NO: 8)
AHAGAAPW;
xvii) a polypeptide having at least 70% sequence identity (e.g. at least 70%, 74%, 80%, 90%, 91%, 92%, 93%, 94%, 95%, 96%, 97%, 98%, 99%, 100%) to the amino acid sequence selected from the group consisting (i)-(xvi);
xviii) a polypeptide having at least 70% identity to the amino acid sequence of (xv);
and/or having at least 80% identity to any one of (viii)-(xiv); and/or having at least 90% to (vi) and/or (vii);
xix) a variant of the polypeptide having amino acid sequence (i), (ii), (iii), (iv), (v), (vi), (vii), (viii), (ix), (x), (xi), (xii), (xiii) or (xiv), wherein said variant comprises a substitution, deletion, and/or insertion at one or more positions;)
xx) a polypeptide comprising amino acid sequences: (i), (ii), (iii) and (iv).

5. The polypeptide according to any one of the preceding claims, wherein said detoxifying Ochratoxin A (OTA) comprising or consisting of hydrolyzing an amide bond of OTA.

6. The polypeptide according to any one of the preceding claims, wherein said polypeptide having amidohydrolase activity (e.g., EC 3.5.1.14).

7. The polypeptide according to any one of the preceding claims, wherein said polypeptide is one or more of the following:
a) said polypeptide belonging to the M20 Peptidase aminoacylase 1-like protein 2-like amidohydrolase subfamily (e.g., M20 Peptidase ACY1L2 amidohydrolase subfamily, e.g., having identifier cd05672 according to the Conserved Domain Database), preferably said M20 Peptidase ACY1L2 amidohydrolase subfamily is characterized by having a metal (e.g. Zn) binding site, further preferably said metal binding site comprising an amino acid sequence motif:
(SEQ ID NO: 21)
CXHXXXXXXXXXXXXXXXXXXXXXXXXXXXXXXXXE;
and/or
b) said polypeptide comprising: an amino acid C or an equivalent amino acid at a position corresponding to position 123 of SEQ ID NO: 1; an amino acid H or an equivalent amino acid at a position corresponding to position 125 of SEQ ID NO: 1; an amino acid E or an equivalent amino acid at a position corresponding to position 160 and/or 161 of SEQ ID NO: 1, preferably at a position corresponding to position 161 of SEQ ID NO: 1; an amino acid H or an equivalent amino acid at a position corresponding to position 185 of SEQ ID NO: 1; and an amino acid H or K, preferably H, or an equivalent amino acid at a position 383 of SEQ ID NO: 1; preferably using the numbering of SEQ ID NO: 1.

8. A polynucleotide, nucleic acid construct or expression vector encoding and/or capable of expressing one or more polypeptides according to any one of the preceding claims.

9. A recombinant host cell (e.g., an isolated recombinant host cell), spore, transgenic plant, transgenic seed or transgenic pollen grain comprising one or more of the following:
(a′) one or more polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
(b′) one or more polynucleotides encoding and/or capable of expressing one or more polypeptides according to (a′);
(c′) one or more nucleic acid constructs and/or expression vectors encoding and/or capable of expressing one or more polypeptides according to (a′).

10. A foodstuff, intermediate foodstuff; fodder, intermediate fodder; feed, intermediate feed;
additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof, comprising one or more of the following:
(a′) one or more polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
(b′) one or more polynucleotides encoding and/or capable of expressing one or more polypeptides according to (a′);
(c′) one or more nucleic acid constructs and/or expression vectors encoding and/or capable of expressing one or more polypeptides according to (a′); and/or
(d′) one or more recombinant host cells, spores, transgenic plants, transgenic seeds and/or transgenic pollen grains comprising one or more of the following: (i′) one or more polypeptides according to (a′); (ii′) one or more polynucleotides according to (b′); and/or (iii′) one or more nucleic acid constructs and/or expression vectors according to (c′); preferably said one or more polypeptides are one or more recombinant and/or isolated polypeptides.

11. A composition or kit comprising one or more of the following:
(a′) polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
(b′) polynucleotides encoding and/or capable of expressing one or more polypeptides according to (a′);
(c′) nucleic acid constructs and/or expression vectors encoding and/or capable of expressing one or more polypeptides according to (a′);
(d′) recombinant host cells, spores, transgenic plants, transgenic seeds and/or transgenic pollen grains, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive);
detoxifying agent, intermediate detoxifying agent; nutritional supplement, intermediate nutritional supplement, prebiotic, intermediate prebiotic and/or mixture/s thereof according to any one of the preceding claims.

12. A foodstuff, intermediate foodstuff; fodder, intermediate fodder; feed, intermediate feed;
additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic and/or mixture/s thereof produced by the method according to any one of the preceding claims, wherein said foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive);
detoxifying agent, intermediate detoxifying agent; nutritional supplement, intermediate nutritional supplement; prebiotic, intermediate prebiotic and/or mixture/s thereof comprising:
(a′) one or more polypeptides capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following;
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
(b′) one or more polynucleotides encoding and/or capable of expressing one or more polypeptides according to (a′);
(c′) one or more nucleic acid constructs and/or expression vectors encoding and/or capable of expressing one or more polypeptides according to (a′); and/or
(d′) one or more recombinant host cells, spores, transgenic plants, transgenic seeds and/or transgenic pollen grains comprising one or more of the following: (i′) one or more polypeptides according to (a′); (ii′) one or more polynucleotides according to (b′); and/or (iii′) one or more nucleic acid constructs and/or expression vectors according to (c′); preferably said one or more polypeptides are one or more recombinant and/or isolated polypeptides.

13. The polypeptide (a′), wherein said polypeptide capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following:
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
polynucleotide encoding and/or capable of expressing said polypeptides according to (a′), nucleic acid construct, expression vector encoding and/or capable of expressing one or more polypeptides according to (a′);
recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding claims;
for use in therapy, prophylaxis and/or as a medicament (e.g., for veterinary use).

14. The polypeptide (a′), wherein said polypeptide capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following:
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
polynucleotide encoding and/or capable of expressing said polypeptide according to (a′), nucleic acid construct, expression vector encoding and/or capable of expressing said polypeptide according to (a′);
recombinant host cell, spore, transgenic plant, transgenic seed, transgenic pollen grain, foodstuff, intermediate foodstuff; fodder, intermediate fodder; feed, intermediate feed;
additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding claims;
for use in one or more of the following methods:
(i) method for treatment, amelioration, prophylaxis and/or diagnostics of mycotoxicosis, preferably an OTA mycotoxicosis;
(ii) method for monitoring development of mycotoxicosis and/or assessing the efficacy of a mycotoxicosis prophylaxis and/or therapy, preferably an OTA mycotoxicosis prophylaxis and/or therapy;
(iii) method for detoxifying ochratoxin A (OTA) and/or altering toxicity of OTA and/or at least one OTA derivative, wherein said OTA derivative is preferably selected from the group consisting of Ochratoxin B and Ochratoxin C;
(iv) method of producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic; pharmaceutical, veterinary, diagnostic, detoxifying, monitoring and/or screening composition or kit;
(v) method according to any one of the preceding claims;
(vi) any combination of methods according to (i)-(v);
(vii) method of any of (i)-(vi), wherein said method is an in vitro, ex vivo or in vivo method.

15. Use of one or more of the following:
(a′) polypeptide capable of detoxifying Ochratoxin A (OTA) and/or at least one OTA derivative, wherein said OTA derivative is preferably selected from the group consisting of Ochratoxin B and Ochratoxin C, wherein said polypeptide is one or more of the following:
(aa′) a polypeptide having at least 64% (e.g., at least 69% or at least 80% or at least 85% or at least 95%, preferably at least 69%) sequence identity to the amino acid sequence selected from the group consisting of: SEQ ID NO: 1, SEQ ID NO: 2, SEQ ID NO: 3 and SEQ ID NO: 4; preferably said polypeptide having an amino acid sequence selected from the group consisting of: SEQ ID NOs: 1-3; further preferably said polypeptide having SEQ ID NO: 1;
(aaa′) a polypeptide comprising one or more of the following amino acid sequences (e.g., conservative motifs):
i)
(SEQ ID NO: 5)
GHACGHNLIA;
ii)
(SEQ ID NO: 6)
IGSPAEEGGGGK;
iii)
(SEQ ID NO: 7)
MAHP;
iv)
(SEQ ID NO: 23)
VNHT;
v)
(SEQ ID NO: 9)
KVAKGLA;
vi)
(SEQ ID NO: 10)
IGSPAEEGGGGKVIL;
vii)
(SEQ ID NO: 11)
AHAGAAPWLG;
viii)
(SEQ ID NO: 12)
PELGFHEVKA;
ix)
(SEQ ID NO: 13)
TNPTSLPTAFVAT;
x)
(SEQ ID NO: 14)
GGRTFGFNAEYDAL;
xi)
(SEQ ID NO: 15)
GHACGHNLIAIVAVASA;
xii)
(SEQ ID NO: 16)
IPGTIKVIGSPAEEGGGGKVILLNEG;
xiii)
(SEQ ID NO: 17)
DACVMAHPEGG;
xiv)
(SEQ ID NO: 18)
AHAGAAPWLGVNALDAAVQGY;
xv)
(SEQ ID NO: 19)
GHACGHNLIAXXAVAXAXGXKAAXXXXXIXGTXKXIGS
PAEEGGGGKXILXNEGXYDXXDACXMAHPXGG,
wherein X is any amino acid;
xvi)
(i), (ii), (iii) and (iv);
and/or
xvii)
(SEQ ID NO: 8)
AHAGAAPW;
(b′) polynucleotide encoding and/or capable of expressing said polypeptide according to (a′) or nucleic acid construct or expression vector encoding and/or capable of expressing said polypeptide according to (a′),
(d′) recombinant host cell, spore, transgenic plant, transgenic seed, transgenic pollen grain, foodstuff, intermediate foodstuff; fodder, intermediate fodder; feed, intermediate feed; additive (e.g., foodstuff-, fodder- or feed additive), intermediate additive (e.g., foodstuff, fodder- or feed intermediate additive); detoxifying agent, intermediate detoxifying agent; nutritional supplement, intermediate nutritional supplement; prebiotic, intermediate prebiotic or mixture/s thereof, composition or kit according to any one of the preceding claims;
for/in one or more of the following:
(i) treatment, amelioration, prophylaxis and/or diagnostics of mycotoxicosis, preferably OTA mycotoxicosis;
(ii) monitoring development of mycotoxicosis and/or assessing the efficacy of a mycotoxicosis prophylaxis and/or therapy, preferably OTA mycotoxicosis prophylaxis and/or therapy;
(iii) detoxifying ochratoxin A (OTA) and/or altering toxicity of OTA and/or at least one OTA derivative, wherein said OTA derivative is preferably selected from the group consisting of Ochratoxin B and Ochratoxin C;
(iv) producing one or more of the following: foodstuff, intermediate foodstuff; fodder, intermediate fodder; feed intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic;
pharmaceutical, veterinary, diagnostic, detoxifying, monitoring and/or screening composition or kit;
(v) in method according to any one of the preceding claims;
(vi) any combination of according to (i)-(v);
(vii) use of any of (i)-(vi), wherein said use is an in vitro, ex vivo or in vivo use.
